SEQUENCE LISTING (14 sequences)

SEQ ID NO: 1 - DNA, 14 bp, synthetic construct
  source 1..14: mol_type = other DNA
accggttcactgtg

SEQ ID NO: 2 - DNA, 14 bp, synthetic construct
  source 1..14: mol_type = other DNA
gaccggttcactgt

SEQ ID NO: 3 - DNA, 16 bp, synthetic construct
  source 1..16: mol_type = other DNA
agaccggttcactgtg

SEQ ID NO: 4-6 - skipped (3 entries)

SEQ ID NO: 7 - RNA, 20 nt, Homo sapiens
  source 1..20: mol_type = transcribed RNA
tcacagtgaaccggtctctt

SEQ ID NO: 8 - DNA, 82 bp, Homo sapiens
  source 1..82: mol_type = genomic DNA
tgagctgttggattcggggccgtagcactgtctgagaggtttacatttctcacagtgaaccggtctctttttcagctgcttc

SEQ ID NO: 9 - RNA, 82 nt, Homo sapiens
  source 1..82: mol_type = transcribed RNA
tgagctgttggattcggggccgtagcactgtctgagaggtttacatttctcacagtgaaccggtctctttttcagctgcttc

SEQ ID NO: 10 - DNA, 21 bp, Homo sapiens
  source 1..21: mol_type = genomic DNA
tcacagtgaaccggtctcttt

SEQ ID NO: 11 - RNA, 21 nt, Homo sapiens
  source 1..21: mol_type = transcribed RNA
tcacagtgaaccggtctcttt

SEQ ID NO: 12 - DNA, 84 bp, Homo sapiens
  source 1..84: mol_type = genomic DNA
tgtgcagtgggaaggggggccgatacactgtacgagagtgagtagcaggtctcacagtgaaccggtctctttccctactgtgtc

SEQ ID NO: 13 - RNA, 84 nt, Homo sapiens
  source 1..84: mol_type = transcribed RNA
tgtgcagtgggaaggggggccgatacactgtacgagagtgagtagcaggtctcacagtgaaccggtgtgtttgggtactgtgct

SEQ ID NO: 14 - DNA, 11 bp, synthetic construct
  source 1..11: mol_type = other DNA
ggttcactgtg

(19) United States
(12) Patent Application Publication
(10) Pub. No.: US 2026/0183329 A1
(43) Pub. Date: Jul. 2, 2026

(54) Inhibitory Nucleic Acids and Methods of Use Thereof

(71) Applicants: The Regents of the University of California, Oakland, CA (US); Aalborg Universitet, Aalborg Øst (DK)

(72) Inventors: Anders Michael Näär, Oakland, CA (US); Caslin Gilroy, Oakland, CA (US); Lei Xu, Danville, CA (US); Sakari Kauppinen, Holte (DK); Marianne Bengtson Løvendorf, København NV (DK); Andreas Petri, Frederiksberg C (DK)

(21) Appl. No.: 19/129,969

(22) PCT Filed: Nov. 20, 2023
(86) PCT No.: PCT/US2023/080512

§ 371 Date: May 14, 2025

Related U.S. Application Data
(60) Provisional application No. 63/427,254, filed on Nov. 22, 2022.

(51) Int. Cl. A61K 31/712; A61K 9/51; A61K 31/7115; A61K 31/7125; A61K 38/28; A61P 3/10; C12N 15/113

(52) U.S. Cl. CPC A61K 31/712; A61K 9/5123; A61K 31/7115; A61K 31/7125; A61K 38/28; A61P 3/10; C12N 15/113; C12N 2310/14; C12N 2310/31; C12N 2310/3231; C12N 2310/334

(57) ABSTRACT

The present disclosure provides inhibitory nucleic acids, compositions comprising the inhibitory nucleic acids, and methods of using the inhibitory nucleic acids to treat various disorders.

### CROSS-REFERENCE

This application claims the benefit of U.S. Provisional Patent Application No. 63/427,254, filed Nov. 22, 2022, which application is incorporated herein by reference in its entirety.

### STATEMENT REGARDING FEDERALLY SPONSORED RESEARCH

This invention was made with government support under DK114277 awarded by the National Institutes of Health. The government has certain rights in the invention.

### INCORPORATION-BY-REFERENCE OF MATERIAL ELECTRONICALLY SUBMITTED

A Sequence Listing is provided herewith as a Sequence Listing XML, “BERK-478WO_SEQ_LIST” created on Nov. 10, 2023 and having a size of 11,512 bytes. The contents of the Sequence Listing XML are incorporated by reference herein in their entirety.

### INTRODUCTION

Metabolic disorders such as insulin resistance, hyperglycemia, type 2 diabetes mellitus, obesity, fatty liver disease, glucose intolerance, hyperinsulinemia, metabolic syndrome, and hypertension, are major health problems.

### SUMMARY

The present disclosure provides inhibitory nucleic acids, compositions comprising the inhibitory nucleic acids, and methods of using the inhibitory nucleic acids to treat various disorders.

### BRIEF DESCRIPTION OF THE DRAWINGS

FIG. 1A-1B provide nucleotide sequences of exemplary antisense oligonucleotides (ASOs) (FIG. 1A; NRC0090, NRC0091, and NRC0119 nucleotide sequences are SEQ ID NOs: 1, 2 and 3, respectively) and the base-pairing location of the ASOs within a target microRNA (miR-128-3p) (FIG. 1B; SEQ ID NO:7).

FIG. 2 schematically depicts effects of dystrophin loss in Duchenne Muscular Dystrophy (DMD).

FIG. 3A-3H depict in vivo effects of miR-128-3p-targeting locked nucleic acid (LNA) antisense oligonucleotides (ASOs).

FIG. 4A-4I depict the effect of miR-128-3p-targeting LNA ASOs on miR-128-3p levels in various tissues in vivo.

FIG. 5A-5F depict the effect of a miR-128-3p-targeting LNA ASO on body weight, body mass, and blood glucose levels.

FIG. 6A-6D depict genetic linkage of elevated circulating mature miR-128 (3p) in DMD mouse model and dog models, and miR-128-1 to weak hand grip strength and pulmonary weakness.

FIG. 7A-7E depict whole body or muscle specific miR-128-3p knockout response in mdx^{5cv} mice.

FIG. 8A-8C depict in vivo ASO dose response in mdx^{5cv} mice.

FIG. 9A-9N depict the effect of a miR-128-1 LNA ASO (“anti-miR-128-1 ASO”) on pathological phenotypes in the mdx^{5cv} DMD mouse model at 6 weeks or 9 weeks of age and depict the effect of a miR-128-1 LNA ASO on muscle fiber sizes, necrosis, and fibrosis.

FIG. 10A-10F depict the effect of ASO administration on indicators of liver and kidney function.

FIG. 11A-11K depict RNAseq analysis of WT mice and mdx5cv mice treated with control ASO and anti-miR-128-1 ASO and depict the effect of a miR-128-1 LNA ASO on dystrophic muscle fiber-type switching in mdx^{5cv} mice.

FIG. 12A-12C depict metabolic analysis of WT mice and mdx5cv mice treated with control ASO and anti-miR-128-1 ASO.

FIG. 13A-13F depict the effect of a miR-128-1 LNA ASO on mitochondrial health in mdx^{5cv} mice.

FIG. 14A-14E depict the effect of a miR-128-1 LNA ASO on inflammation, ferroptosis, and oxidative stress.

FIG. 15A-15D depict histology of small intestine sections of wild-type (WT) and mdx^{5cv} mice treated with control ASO or anti-miR-128-1 ASO.

FIG. 16A-16B depict evaluation of miR-128 expression and its downstream targets in the cardiac muscle of mdx^{5cv} mice at 6 weeks of age.

FIG. 17A-17L depict the effect of anti-miR-128-1 ASO in a myocardial infarction (MI) mouse model and preclinical DMD^{Y/-} pig model.

FIG. 18A-18C depict the effect of anti-miR-128-1 ASO on fibrosis in the MI mouse model.

### DEFINITIONS

As used herein, an “antisense oligonucleotide” refers to a nucleic acid sequence that is complementary to a DNA or RNA sequence, such as that of a microRNA.

“RNA” refers to a molecule comprising at least one or more ribonucleotide residues. A “ribonucleotide” is a nucleotide with a hydroxyl group at the 2′ position of a beta-D-ribofuranose moiety. The term RNA, as used herein, includes double-stranded RNA, single-stranded RNA, isolated RNA, such as partially purified RNA, essentially pure RNA, synthetic RNA, recombinantly produced RNA, as well as altered RNA that differs from naturally occurring RNA by the addition, deletion, substitution and/or alteration of one or more nucleotides. Nucleotides of the RNA molecules can also comprise non-standard nucleotides, such as non-naturally occurring nucleotides or chemically synthesized nucleotides or deoxynucleotides.

A “microRNA” (miRNA) is a single-stranded RNA molecule of about 21-23 nts in length. In general, miRNAs regulate gene expression. miRNAs are encoded by genes from whose DNA they are transcribed, but miRNAs are not translated into protein. Each primary miRNA transcript is processed into a short stem-loop structure before undergoing further processing into a functional miRNA. Mature miRNA molecules are partially complementary to one or more messenger RNA (mRNA) molecules, and their main function is to down-regulate gene expression.

As used herein “an interfering RNA” refers to any double stranded or single stranded RNA sequence, capable, either directly or indirectly (i.e., upon conversion), of inhibiting or down regulating gene expression by mediating RNA interference. Interfering RNA includes but is not limited toenail interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense, region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be for example, about 18 to 21 nucleotides long.

As used herein, an “antagomir” refers to a small synthetic RNA having complementarity to a specific microRNA target, with either mispairing at the cleavage site or one or more base modifications to inhibit cleavage.

As used herein, the phrase “post-transcriptional processing” refers to mRNA processing that occurs after transcription and is mediated, for example, by the enzymes Dicer and/or Drosha.

By “an effective amount” is meant the amount of a required agent (e.g., an inhibitory nucleic acid of the present disclosure) or composition (e.g., composition comprising an inhibitory nucleic acid of the present disclosure), comprising the agent to ameliorate the symptoms of a disease relative, to an untreated patient. The effective amount of an agent or a composition used to practice a therapeutic treatment of a disease or disorder varies depending upon the manner of administration, the age, body weight, and general health of the subject. Ultimately, the attending, physician or veterinarian will decide the appropriate amount and dosage regimen. Such amount is referred to as an “effective” amount.”

As used herein, “cholesterol homeostasis” refers to the regulation of cholesterol uptake, cholesterol biosynthesis, cholesterol conversion to bile acids and excretion of bile acids as such processes occur in a subject having healthful levels of LDL, HDL and cholesterol in the blood (e.g., such healthful levels are also referred to herein as a “reference standard”). Accordingly, a subject in need of cholesterol homeostasis is in need of improved regulation resulting in a return to healthful levels of LDL, HDL and/or cholesterol in the blood.

The terms “diabetes” and “diabetic” refer to a progressive disease of carbohydrate metabolism involving inadequate production or utilization of insulin, frequently characterized by hyperglycemia and glycosuria. The terms “pre-diabetes” and “pre-diabetic” refer to a state wherein a subject does not have the characteristics, symptoms and the like typically observed in diabetes, but does have characteristics, symptoms and the like that, if left untreated, may progress to diabetes. The presence of these conditions may be determined using, for example, either the fasting plasma glucose (FPG) test or the oral glucose tolerance test (OGTT). Both usually require a subject to fast for at least 8 hours prior to initiating the test. In the FPG test, a subject's blood glucose is measured after the conclusion of the fasting; generally, the subject fasts overnight and the blood glucose is measured in the morning before the subject eats. A healthy subject would generally have a FPG concentration between about 90 and about 100 mg/dl, a subject with “pre-diabetes” would generally have a FPG concentration between about 100 and about 125 mg/dl, and a subject with “diabetes” would generally have a FPG level above about 126 mg/dl. In the OGTT, a subject's blood glucose is measured after fasting and again two hours after drinking a glucose-rich beverage. Two hours after consumption of the glucose-rich beverage, a healthy subject generally has a blood glucose concentration below about 140 mg/dl, a pre-diabetic subject generally has a blood glucose concentration about 140 to about 199 mg/dl, and a diabetic subject generally has a blood glucose concentration about 200 mg/dl or above. While the aforementioned glycemic values pertain to human subjects, normoglycemia, moderate hyperglycemia and overt hyperglycemia are scaled differently in murine subjects. A healthy murine subject after a four-hour fast would generally have a FPG concentration between about 100 and about 150 mg/dl, a murine subject with “pre-diabetes” would generally have a FPG concentration between about 175 and about 250 mg/dl and a murine subject with “diabetes” would generally have a FPG concentration above about 250 mg/dl.

The term “insulin resistance” as used herein refers to a condition where a normal amount of insulin is unable to produce a normal physiological or molecular response. In some cases, a hyper-physiological amount of insulin, either endogenously produced or exogenously administered, is able to overcome the insulin resistance, in whole or in part, and produce a biologic response.

The term “metabolic syndrome” refers to an associated cluster of traits that includes, but is not limited to, hyperinsulinemia, abnormal glucose tolerance, obesity, redistribution of fat to the abdominal or upper body compartment, hypertension, dysfibrinolysis, and dyslipidemia characterized by high triglycerides, low high density lipoprotein (HDL)-cholesterol, and high small dense low density lipoprotein (LDL) particles. Subjects having metabolic syndrome are at risk for development of Type 2 diabetes and/or other disorders (e.g., atherosclerosis).

The term “glucose metabolism disorder” encompasses any disorder characterized by a clinical symptom or a combination of clinical symptoms that is associated with an elevated level of glucose and/or an elevated level of insulin in a subject relative to a healthy individual. Elevated levels of glucose and/or insulin may be manifested in the following diseases, disorders and conditions: hyperglycemia, type 2 diabetes, gestational diabetes, type 1 diabetes, insulin resistance, impaired glucose tolerance, hyperinsulinemia, impaired glucose metabolism, pre-diabetes, other metabolic disorders (such as metabolic syndrome, which is also referred to as syndrome X), and obesity, among others. The polypeptides of the present disclosure, and compositions thereof, can be used, for example, to achieve and/or maintain glucose homeostasis, e.g., to reduce glucose level in the bloodstream and/or to reduce insulin level to a range found in a healthy subject.

The term “hyperglycemia”, as used herein, refers to a condition in which an elevated amount of glucose circulates in the blood plasma of a subject relative to a healthy individual. Hyperglycemia can be diagnosed using methods known in the art, including measurement of fasting blood glucose levels as described herein.

The term “hyperinsulinemia”, as used herein, refers to a condition in which there are elevated levels of circulating insulin when, concomitantly, blood glucose levels are either elevated or normal. Hyperinsulinemia can be caused by insulin resistance which is associated with dyslipidemia, such as high triglycerides, high cholesterol, high low-density lipoprotein (LDL) and low high-density lipoprotein (HDL); high uric acids levels; polycystic ovary syndrome; type 2 diabetes and obesity. Hyperinsulinemia can be diagnosed as having a plasma insulin level higher than about 2 μU/mL.

As used herein, the phrase “body weight disorder” refers to conditions associated with excessive body weight and/or enhanced appetite. Various parameters are used to determine whether a subject is overweight compared to a reference healthy individual, including the subject's age, height, sex and health status. For example, a subject may be considered overweight or obese by assessment of the subject's Body Mass Index (BMI), which is calculated by dividing a subject's weight in kilograms by the subject's height in meters squared. An adult having a BMI in the range of from about 18.5 kg/m² to about 24.9 kg/m² is considered to have a normal weight; an adult having a BMI between about 25 kg/m² and about 29.9 kg/m² may be considered overweight (pre-obese); and an adult having a BMI of about 30 kg/m² or higher may be considered obese. Enhanced appetite frequently contributes to excessive body weight. There are several conditions associated with enhanced appetite, including, for example, night eating syndrome, which is characterized by morning anorexia and evening polyphagia often associated with insomnia, but which may be related to injury to the hypothalamus

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets. In some cases, an “individual” is a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antisense oligonucleotide” includes a plurality of such oligonucleotides and reference to “the nucleic acid modification” includes reference to one or more nucleic acid modifications and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the present disclosure, which are, for clarity, described in the context of separate embodiments, may also be provided in combination in a single embodiment. Conversely, various features of this disclosure, which are, for brevity, described in the context of a single embodiment, may also be provided separately or in any suitable sub-combination. All combinations of the embodiments pertaining to this disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides inhibitory nucleic acids that reduce the level of miR-128-1 in a cell, and compositions comprising the inhibitory nucleic acids. The present disclosure provides methods of treating various disorders, using an inhibitory nucleic acid of the present disclosure.

### Inhibitory Nucleic Acids

The present disclosure provides inhibitory nucleic acids that reduce the level of miR-128-1 in a cell (e.g., a cell in an individual). In some cases, the inhibitory nucleic acids are antisense oligonucleotides (ASOs). The present disclosure provides ASOs that reduces the level of miR-128-1 in a cell (e.g., in a cell in an individual).

In some cases, an miR-128 comprises the nucleotide sequence: tgagctgttg gattoggggc cgtagcactg tctgagaggt ttacatttct cacagtgaac cggtctcttt ttcagctgct tc (SEQ ID NO:5). In some cases, an miR-128 comprises the nucleotide sequence: ugagcuguug gauucggggc cguagcacug ucugagaggu uuacauuucu cacagugaac cggucucuuu uucagcugcu uc (SEQ ID NO:6). In some cases, an miR-128 comprises the nucleotide sequence: tcacagtgaa ccggtctctt t (SEQ ID NO:10. In some cases, an miR-128 comprises the nucleotide sequence: ucacagugaa ccggucucuu u (SEQ ID NO:11). In some cases, an miR-128 comprises the nucleotide sequence: tgtgcagtgg gaaggggggc cgatacactg tacgagagtg agtagcaggt ctcacagtga accggtctct ttccctactg tgtc (SEQ ID NO: 12). In some cases, an miR-128 comprises the nucleotide sequence: ugugcagugg gaaggggggc cgauacacug uacgagagug aguagcaggu cucacaguga accggugugu uuggguacug ugcu (SEQ ID NO:13).

In some cases, an inhibitory nucleic acid (e.g., an ASO) of the present disclosure reduces the level of miR-128-1 in a cell more potently than an inhibitory nucleic acid (e.g., an ASO) comprising the nucleotide sequence: 5′-TTCACTGTG-3′. In some cases, an inhibitory nucleic acid (e.g., an ASO) of the present disclosure is at least 2-fold, at least 2.5-fold, at least 5-fold, at least 10-fold, at least 15-fold, at least 20-fold, at least 25-fold, at least 30-fold, or at least 40-fold, more potent than an inhibitory nucleic acid (e.g., an ASO) comprising the nucleotide sequence: 5′-TTCACTGTG-3′. For example, in some cases an inhibitory nucleic acid (e.g., an ASO) of the present disclosure reduces the level of miR-128-1 in a cell to an extent that is at least 10%, at least 15%, at least 20%, at least 25%, at least 50%, at least 75%, at least 90%, or more than 90%, greater than the extent to which an inhibitory nucleic acid (e.g., an ASO) comprising the nucleotide sequence 5′-TTCACTGTG-3′ reduces the level of miR-128-1 in the cell, when administered in the same amount as the inhibitory nucleic acid (e.g., an ASO) of the present disclosure. In some cases, an inhibitory nucleic acid (e.g., an ASO) of the present disclosure achieves a reduction of at least 10%, at least 15%, at least 20%, at least 25%, at least 50%, at least 75%, or at least 90%, of the level of miR-128-1 in a cell in an individual when the individual is administered with an inhibitory nucleic acid (e.g., an ASO) of the present disclosure in an amount that is at least 10%, at least 15%, at least 20%, at least 25%, at least 50%, at least 75%, at least 90%, or more than 90%, less than the amount of an inhibitory nucleic acid (e.g., an ASO) comprising the nucleotide sequence: 5′-TTCACTGTG-3′ required to achieve the same reduction in the level of miR-128-1.

In some cases, an inhibitory nucleic acid (e.g., an ASO) of the present disclosure reduces the level of miR-128-1 in a cell more potently than an inhibitory nucleic acid (e.g., an ASO) comprising the nucleotide sequence: 5′-GGTTCACTGTG-3′ (SEQ ID NO:14). In some cases, an inhibitory nucleic acid (e.g., an ASO) of the present disclosure is at least 2-fold, at least 2.5-fold, at least 5-fold, at least 10-fold, at least 15-fold, at least 20-fold, at least 25-fold, at least 30-fold, or at least 40-fold, more potent than an inhibitory nucleic acid (e.g., an ASO) comprising the nucleotide sequence: 5′-GGTTCACTGTG-3′ (SEQ ID NO: 14). For example, in some cases an inhibitory nucleic acid (e.g., an ASO) of the present disclosure reduces the level of miR-128-1 in a cell to an extent that is at least 10%, at least 15%, at least 20%, at least 25%, at least 50%, at least 75%, at least 90%, or more than 90%, greater than the extent to which an inhibitory nucleic acid (e.g., an ASO) comprising the nucleotide sequence 5′-GGTTCACTGTG-3′ (SEQ ID NO:14) reduces the level of miR-128-1 in the cell, when administered in the same amount as the inhibitory nucleic acid (e.g., an ASO) of the present disclosure. In some cases, an inhibitory nucleic acid (e.g., an ASO) of the present disclosure achieves a reduction of at least 10%, at least 15%, at least 20%, at least 25%, at least 50%, at least 75%, or at least 90%, of the level of miR-128-1 in a cell in an individual when the individual is administered with an inhibitory nucleic acid (e.g., an ASO) of the present disclosure in an amount that is at least 10%, at least 15%, at least 20%, at least 25%, at least 50%, at least 75%, at least 90%, or more than 90%, less than the amount of an inhibitory nucleic acid (e.g., an ASO) comprising the nucleotide sequence: 5′-GGTTCACTGTG-3′ (SEQ ID NO:14) required to achieve the same reduction in the level of miR-128-1.

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid (i.e., miR-128) and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112. In some cases, an inhibitory nucleic acid of the present disclosure is an ASO.

In some cases, the inhibitory nucleic acids are 10 to 50, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some cases, an inhibitory nucleic acid of the present disclosure is 15 nucleotides in length. In some cases, an inhibitory nucleic acid of the present disclosure is 12 to 30 or 13 to 30 nucleotides in length. One having ordinary skill in the art will appreciate that this embodies inhibitory nucleic acids having a length of 12, 13, 14, 15, 16, 17, 18, 19, 20, 21, 22, 23, 24, 25, 26, 27, 28, 29 or 30 nucleotides.

In some cases, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the present disclosure may be formed as composite structures of two or more oligonucleotides, modified oligonucleotides, oligonucleosides and/or oligonucleotide mimetics as described above. Such compounds have also been referred to in the art as hybrids or gapmers.

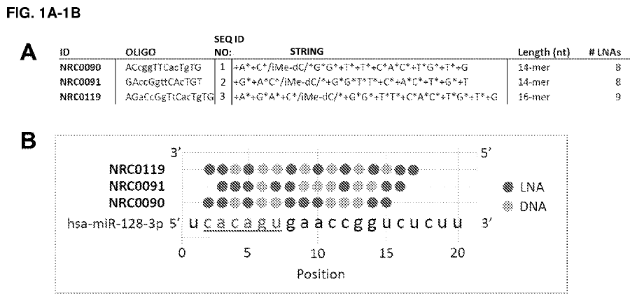

As one non-limiting example, in some cases, a suitable inhibitory nucleic acid comprises the following nucleotide sequence: 5′-ACCGGTTCACTGTG-3′ (SEQ ID NO:1) and has a length of from 14 nucleotides to 20 nucleotides. In some cases, the inhibitory nucleic acid comprises the following nucleotide sequence: 5′-ACCGGTTCACTGTG-3′ (SEQ ID NO:1) and has a length of 14 nucleotides. In some cases, the inhibitory nucleic acid comprises one or more of: i) a locked nucleic acid (LNA); ii) a modified backbone; and iii) a 5-methyl deoxycytosine. In some cases, a suitable inhibitory nucleic acid comprises the nucleotide sequence, and all of the modifications, depicted in FIG. 1A and designated “NRC0090.” The ASO referred to in FIG. 1A as NRC0090 is: +A*+C*/iMe-dC/*G*G*+T*+T*+C*A*C*+T*G*+T*+G, where “+” precedes an LNA; * precedes a phosphorothioated base, and/iMe-dC/ denotes an internal 5-methyl deoxycytosine.

As another non-limiting example, in some cases, a suitable inhibitory nucleic acid comprises the following nucleotide sequence: 5′-GACCGGTTCACTGT-3′ (SEQ ID NO:2) and has a length of from 14 nucleotides to 20 nucleotides. In some cases, the inhibitory nucleic acid comprises the following nucleotide sequence: 5′-GACCGGTTCACTGT-3′ (SEQ ID NO:2) and has a length of 14 nucleotides. In some cases, the inhibitory nucleic acid comprises one or more of: i) a locked nucleic acid (LNA); ii) a modified backbone; and iii) a 5-methyl deoxycytosine. In some cases, a suitable inhibitory nucleic acid comprises the nucleotide sequence, and all of the modifications, depicted in FIG. 1A and designated “NRC0091.” The ASO referred to in FIG. 1A as NRC0091 is +G*+A*C*/iMe-dC/*+G*G*T*T*+C*+A*C*+T*+G*+T, where “+” precedes an LNA; * precedes a phosphorothioated base, and/iMe-dC/ denotes an internal 5-methyl deoxycytosine.

As another non-limiting example, in some cases, a suitable inhibitory nucleic acid comprises the following nucleotide sequence: 5′-AGACCGGTTCACTGTG-3′ (SEQ ID NO:3) and has a length of from 14 nucleotides to 20 nucleotides. In some cases, the inhibitory nucleic acid comprises the following nucleotide sequence: AGACCGGTTCACTGTG-3′ (SEQ ID NO:3) and has a length of 14 nucleotides. In some cases, the inhibitory nucleic acid comprises one or more of: i) a locked nucleic acid (LNA); ii) a modified backbone; and iii) a 5-methyl deoxycytosine. In some cases, a suitable inhibitory nucleic acid comprises the nucleotide sequence, and all of the modifications, depicted in FIG. 1A and designated “NRC0119.” The ASO referred to in FIG. 1A as NRC0119 is +A*+G*A*+C*/iMe-dC/*+G*G*+T*T*+C*A*C*+T*G*+T*+G, where “+” precedes an LNA; * precedes a phosphorothioated base, and/iMe-dC/ denotes an internal 5-methyl deoxycytosine.

In some embodiments, the inhibitory nucleic acid comprises at least one nucleotide modified at the 2′ position of the sugar, e.g., a 2′-O-alkyl, 2′-O-alkyl-O-alkyl or 2′-fluoro-modified nucleotide. In other cases, RNA modifications include 2′-fluoro, 2′-amino and 2′O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than: 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. In some cases, inhibitory nucleic acids are oligonucleotides with phosphorothioate backbones and those with heteroatom backbones, particularly CH₂—NH—O—CH₃, CH₃—N(CH₃)—O—CH₂ (known as a methylene (methylimino) or MMI backbone], CH₂—O—N(CH₃)—CH₂, CH₂—N(CH₃)—N(CH₃)—CH₂ and O—N(CH₃)—CH₂—CH₂ backbones, wherein the native-phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters; aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide; sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

One or more substituted sugar moieties can also be included, e.g., one of the following at the 2′ position: OH, SH, SCH₃, F, OCN, OCH₃OCH₃, OCH₃, O(CH₂) n CH₃, O(CH₂)nNH₂ or O(CH₂)nCH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A suitable modification includes 2′-methoxyethoxy [2′-O—CH₂CH₂OCH₃, also known as: 2′-O-(2-methoxyethyl)] (Martin et al, Helv. Chim. Acta, 1995, 78, 486). Other modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cylosine, (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine and 2,6-diaminopurine. A “universal” base known in the art, e.g., inosine, can also be included. 5-Me-C substitutions have been shown to increase nucleic acid duplex stability by 0.6-12<0>C. In some cases, an inhibitory nucleic acid of the present disclosure comprises one or more 5-Me-C.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by English et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of an inhibitory nucleic acid. These include 5-substituted pyrimidines, 6-azapyrimidines and N-2, N-6 and 0-6 substituted purines, comprising 2-aminopropyladenine, 5-propynyluracil and 5-propynylcytosine, 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are suitable for inclusion in an inhibitory nucleic acid, e.g., alone or together with 2′-O-methoxyethyl sugar modifications.

In some cases, the inhibitory nucleic acids are chemically linked to one or more, moieties or conjugates that enhance the activity, cellular distribution, or cellular uptake of the oligonucleotide. Such moieties comprise but are not limited to, lipid moieties such as a cholesterol moiety; cholic acid; a thioether, e.g., hexyl-S-tritylthiol; a thiocholesterol; an aliphatic chain, e.g., dodecandiol or undecyl residues; a phospholipid; e.g., di-hexadecyl-rac-glycerol or triethylammonium 1,2-di-O-hexadecyl-rac-glycero-3-H-phosphonate; a polyamine or a polyethylene glycol chain; adamantane acetic acid; a palmityl moiety; an octadecylamine moiety; or a hexylamino-carbonyl-t oxycholesterol moiety.

These moieties or conjugates can include conjugate groups covalently bound to functional groups such as primary or secondary hydroxyl groups. Conjugate groups suitable for use include intercalators, importer molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation and/or strengthen sequence-specific hybridization with the target nucleic acid. Groups that enhance the pharmacokinetic properties include groups that improve uptake, distribution, metabolism or excretion of an inhibitory nucleic acid. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety.

The inhibitory nucleic acids useful in the present methods are sufficiently complementary to all or part of miR-128, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base, al one position of an inhibitory nucleic acid is capable of hydrogen bonding with a base at the corresponding position of a miR-128 sequence, then the bases are considered to be complementary to each other at that position. 100% complementary to is not required.

In the context of this disclosure, hybridization, means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. The inhibitory nucleic acids and the miR-128 are complementary to each other when a sufficient number of corresponding positions in each molecule are occupied, by nucleotides that can hydrogen bond with each other. Thus, “specifically hybridizable” and “complementary” are terms which are used to indicate a sufficient degree of complementarity or precise pairing such that stable and specific binding occurs between the inhibitory nucleic acid and the miR-128 target sequence. For example, if a base at one position of an inhibitory nucleic acid is capable of hydrogen bonding with a base at the corresponding position of a miR-128 molecule; then the bases are considered complementary to each other at that position.

Although in some embodiments, 100% complementarity is desirable, it is understood in the art that a complementary nucleic acid sequence need not be 100% complementary to that of its target nucleic acid to be specifically, hybridizable. A complementary nucleic acid sequence for purposes of the present methods is specifically hybridizable when binding of the sequence to the target miR-128 molecule interferes with the normal function of the target miR-128 to cause a loss of activity, and there is a sufficient degree of complementarity to avoid non-specific binding of the sequence to non-target miR-128 sequences under conditions in which specific binding is desired, e.g., under physiological conditions in the case of in vivo assays or therapeutic treatment, and in the case of in vitro assays, under conditions in which the assays are performed under suitable conditions, of stringency. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50%, formamide. Stringent temperature conditions will ordinarily include temperatures of at least 30 degrees C., at least about 37 degrees C., or at least about 42 degrees C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. As a non-limiting example, in some cases, hybridization will occur at 30 degrees C. in 750 mM NaCl, 75 mM trisodium citrate, and 1% SDS. As another non-limiting example, in some cases, hybridization will occur at 37 degrees C. in 500 mM NaCl, 50 mM trisodium citrate, 1% SDS, 35% formamide, and 100 μg/ml denatured salmon sperm DNA (ssDNA). As another non-limiting example, in some cases, hybridization will occur at 42 degrees C. in 250 mM NaCl, 25 mM trisodium citrate, 1% SDS, 50% formamide, and 200 μg/ml ssDNA. Useful variations on these conditions will be readily apparent to those skilled in the art.

In general, the inhibitory nucleic acids useful in the methods described herein have at least 80% sequence complementarity to a target region within the target nucleic acid, e.g., 90%, 95%, or 100% sequence complementarity to the target region within miR-128 (e.g., a target region comprising the seed sequence). For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). An inhibitory nucleic acid (e.g., an ASO) that hybridizes to a miR-128 target sequence can be identified through routine experimentation. In general, the inhibitory nucleic acids must retain specificity for their target, i.e., must not directly bind to, or directly significantly affect expression levels of transcripts other than the intended target.

### Antisense

As noted above, in some cases, an inhibitory nucleic acid of the present disclosure are ASOs. ASOs are typically designed to block expression of a DNA or RNA target by binding to the target and halting expression at the level of transcription, translation, or splicing. ASOs of the present disclosure are complementary nucleic acid sequences designed to hybridize under stringent conditions to a miR-128 target sequence. Thus, oligonucleotides are chosen that are sufficiently complementary to the target, i.e., that hybridize sufficiently well and with sufficient specificity, to give the desired effect.

### Modified Bases/Locked Nucleic Acids (LNAs)

In some cases, an inhibitory nucleic acid of the present disclosure comprises one or more modified bonds or bases. Modified bases include phosphorothioate, methylphosphonate, peptide nucleic acids, or locked nucleic acid (LNA) molecules. For example, in some cases, the modified nucleotides are locked nucleic acid molecules, including [alpha]-L-LNAs. LNAs comprise ribonucleic acid analogues wherein the ribose ring is “locked” by a methylene bridge between the 2′-oxygen and the 4′-carbon—i.e., oligonucleotides, containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a miR-128 target sequence. The LNA molecules can be chemically synthesized using methods known in the art.

### Antagomirs

In some cases, the inhibitory nucleic acid is an antagomir. Antagomirs are chemically modified antisense oligonucleotides that target a miR-128 target sequence. For example, an antagomir for use in the methods described herein can include a nucleotide sequence sufficiently complementary to hybridize to a miR-128 target sequence of about 12 to 25 nucleotides, or from about 15 to 23 nucleotides.

In general, antagomirs include a cholesterol moiety, e.g., at the 3′-end. In some embodiments, antagomirs have various modifications for RNase protection and pharmacologic properties such as enhanced tissue and cellular uptake. For example, in addition to the modifications discussed above for antisense oligos, an antagomir can have one or more of complete or partial 2′-O-methylation of sugar and/or a phosphorothioate backbone. Phosphorothioate modifications provide protection against RNase activity and their lipophilicity contributes to enhanced tissue uptake. In some embodiments, the antagomir can include six phosphorothioate backbone modifications; two phosphorothioates are located at the 5′-end and four at the 3′-end. Antagomirs useful in the present methods can also be modified with respect to their length or otherwise the number of nucleotides making up the antagomir. The antagomirs must retain specificity for their target, i.e., must not directly bind to, or directly significantly affect expression levels of, transcripts other than the intended target. In some embodiments, the inhibitory nucleic acid is locked and includes a cholesterol moiety (e.g., a locked antagomir).

siRNA/shRNA

In some cases, an inhibitory nucleic acid of the present disclosure is an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic-acid sequence or a portion thereof, and wherein the circular polynucleotide can be processed either in vivo or in vitro to generate an active siRNA molecule capable of mediating RNA interference.

In some cases, the interfering RNA coding region encodes a self-complementary RNA molecule having a sense region, an antisense region and a loop region. Such an RNA molecule when expressed desirably forms a “hairpin” structure, and is referred to herein as an “shRNA,” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology.

The target RNA cleavage reaction guided by siRNAs is highly sequence specific. In general, siRNA containing a nucleotide sequence identical to a portion of the target nucleic acid are used for inhibition. However, 100% sequence identity between the siRNA and the target gene is not required. Thus, the present disclosure has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general, the siRNAs must retain specificity for their target, i.e., must not directly bind to, or directly significantly affect expression levels of, transcripts other than the intended target.

### Making Inhibitory Nucleic Acids

Inhibitory nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

### Pharmaceutical Compositions

The present disclosure provides compositions, including pharmaceutical compositions, comprising an inhibitory nucleic acid of the present disclosure. An inhibitory nucleic acid of the present disclosure may be referred to below as an “agent” or an “active agent.”

In some cases, the compositions are formulated with a pharmaceutical acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). An inhibitory nucleic acid can be formulated for administration, in any convenient way for use in human or veterinary medicine. Wetting agents, emulsifiers and lubricants, such as sodium lauryl sulfate and magnesium stearate, as well as coloring agents release agents, coating agents, sweetening, flavoring and perfuming agents, preservatives and antioxidants can also be present in the compositions.

Formulations of an inhibitory nucleic acid of the present disclosure include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount, of active ingredient (e.g., an inhibitory nucleic acid of the present disclosure) which can be combined with a carrier material to produce a single dosage form will vary depending upon the host being treated, the particular mode of administration, e.g.; intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixed with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., an inhibitory nucleic acid of the present disclosure) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more Sweetening agents, such as sucrose, aspartame or saccharin. Formulations can be adjusted for osmolarity.

In some cases, oil-based pharmaceuticals are used for administration of an inhibitory nucleic acid. Oil-based suspensions can be formulated by suspending ah active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, an injectable oil-in-water emulsion comprises a paraffin oil, a sorbitan monooleate, an ethoxylated sorbitan monooleate and/or an ethoxylated sorbitan trioleate.

The pharmaceutical compositions can be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some cases, the pharmaceutical compositions can be delivered transdermally, by a topical route, formulated as applicator sticks, solutions, suspensions, emulsions, gels, creams, ointments, pastes jellies, paints, powders, and aerosols.

In some cases, the pharmaceutical compositions can be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some cases, the pharmaceutical compositions can be parenterally administered, such as by intravenous (IV) administration or administration into a body cavity or lumen of an organ. These formulations can comprise a solution of active agent (e.g., an inhibitory nucleic acid of the present disclosure) in a pharmaceutically acceptable carrier. Acceptable vehicles and solvents that can be employed are water and Ringer's solution, an isotonic sodium chloride. In addition, sterile fixed, oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For intravenous administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally acceptable diluent or solvent, such as a solution of 1,3-butanediol. The administration can be by bolus or continuous infusion (e.g., substantially uninterrupted introduction into a blood vessel for a specified period of time).

In some cases, the pharmaceutical composition can be lyophilized. Stable lyophilized compositions comprising an inhibitory nucleic acid can be made by lyophilizing a solution comprising a pharmaceutical composition of the present disclosure and a bulking agent, e.g., mannitol, trehalose, raffinose, and sucrose or mixtures thereof. A process for preparing a stable lyophilized formulation can include lyophilizing a solution of about 2.5 mg/mL nucleic acid, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent (e.g., an inhibitory nucleic acid of the present disclosure) into target cells w vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used herein, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the present disclosure can be administered for prophylactic and/or therapeutic treatments. In some cases, for therapeutic applications, compositions are administered to a subject in need thereof (e.g., an individual who is at risk of (e.g., at greater risk than the general population) or has a disorder described herein) in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use. i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Remington: The Science and Practice of Pharmacy, 21st ed., 2005). The state of the art allows the clinician to determine the dosage regimen for each individual patient, active agent (e.g., inhibitory nucleic acid) and disease or condition treated. Guidelines provided for similar compositions used as pharmaceuticals can be used as guidance to determine the dosage regiment, i.e., dose schedule and dosage levels.

Single or multiple administrations of formulations can be given depending on for example: the dosage and frequency as required and tolerated by the patient, the degree and amount of therapeutic effect generated after each administration (e.g., effect on blood glucose levels), and the like. The formulations should provide a sufficient quantity of active agent (e.g., inhibitory nucleic acid) to effectively treat, prevent or ameliorate conditions, diseases or symptoms.

In alternative embodiments, pharmaceutical formulations for oral administration are in a daily amount of between about 1 μg to about 100 mg nucleic acid per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

In some embodiments, the methods described herein can include co-administration with other drugs or pharmaceuticals, e.g., compositions for reducing blood glucose levels. For example, the inhibitory nucleic acids can be co-administered with drugs for treating or reducing risk of a disorder described herein.

### Methods of Treatment—Metabolic Disorders

The present disclosure provides methods using an inhibitory nucleic acid of the present disclosure, e.g., in subjects suffering from Metabolic Syndrome (e.g., high LDL, low HDL, high triglycerides, obesity, nonalcoholic fatty liver disease, insulin resistance, and/or hypertension), type 2 diabetes, and/or cardiovascular disease (CVD). A treatment method of the present disclosures provides for one or more of the following: a) lowering of circulating LDL; b) increased HDL; c) lowered triglycerides; d) decreased obesity; e) decreased insulin resistance and type 2 diabetes; f) ameliorated nonalcoholic fatty liver disease; and g) decreased atherosclerosis/CVD. The methods comprise administering to an individual in need thereof an effective amount of an inhibitory nucleic acid of the present disclosure, or a composition (e.g., a pharmaceutical composition) comprising same. In some cases, an inhibitory nucleic acid of the present disclosure is administered in a lipid nanoparticle. In some cases, an inhibitory nucleic acid of the present disclosure is administered in a liposome.

A “therapeutically effective amount” refers to the administration of an agent (e.g., an inhibitory nucleic acid) to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects. In some cases, e.g., in the case of a hyperglycemic condition, a lowering or reduction of blood glucose or an improvement in glucose tolerance test can be used to determine whether the amount of an agent is effective to treat the hyperglycemic condition. The therapeutically effective amount can be adjusted in connection with the dosing regimen and diagnostic analysis of the subject's condition and the like.

In some cases, a therapeutically effective amount of an inhibitory nucleic acid of the present disclosure is an amount that, when administered in one or more doses, is sufficient to reduce or decrease any level (e.g., a baseline level) of fasting plasma glucose (FPG), wherein, for example, the amount is sufficient to reduce a FPG level greater than 200 mg/dl to less than 200 mg/dl, wherein the amount is sufficient to reduce a FPG level between 175 mg/dl and 200 mg/dl to less than the starting level, wherein the amount is sufficient to reduce a FPG level between 150 mg/dl and 175 mg/dl to less than the starting level, wherein the amount is sufficient to reduce a FPG level between 125 mg/dl and 150 mg/dl to less than the starting level, and so on (e.g., reducing FPG levels to less than 125 mg/dl, to less than 120 mg/dl, to less than 115 mg/dl, to less than 110 mg/dl, etc.).

In some cases, a therapeutically effective amount of an inhibitory nucleic acid of the present disclosure is an amount is an amount that, when administered in one or more doses, is sufficient to reduce or decrease hemoglobin A1c (HbA1c) levels by more than about 10% to 9%, by more than about 9% to 8%, by more than about 8% to 7%, by more than about 7% to 6%, by more than about 6% to 5%, and so on. In some cases, a therapeutically effective amount of an inhibitory nucleic acid of the present disclosure is an amount is an amount sufficient to reduce or decrease HbA1c levels by about 0.1%, 0.25%, 0.4%, 0.5%, 0.6%, 0.7%, 0.8%, 0.9%, 1%, 1.5%, 2%, 3%, 4%, 5%, 10%, 20%, 30%, 33%, 35%, 40%, 45%, 50%, or more.

In some cases, a therapeutically effective amount of an inhibitory nucleic acid of the present disclosure is an amount is an amount that, when administered in one or more doses, is sufficient to result in insulin levels in a normal range.

In some cases, a therapeutically effective amount of an inhibitory nucleic acid of the present disclosure is an amount is an amount that, when administered in one or more doses, is sufficient to result in serum alanine transaminase (ALT) levels in a normal range. In some cases, a therapeutically effective amount of an inhibitory nucleic acid of the present disclosure is an amount is an amount that, when administered in one or more doses, is sufficient to reduce serum ALT levels by at least at least about 10%, at least about 20%, at least about 25%, at least about 30%, at least about 40%, at least about 50%, or more than 50%, compared to the serum ALT level before treatment. In some cases, a therapeutically effective amount of an inhibitory nucleic acid of the present disclosure is an amount is an amount that, when administered in one or more doses, is sufficient to result in serum aspartate transaminase (AST) in a normal range. In some cases, a therapeutically effective amount of an inhibitory nucleic acid of the present disclosure is an amount is an amount that, when administered in one or more doses, is sufficient to reduce serum AST levels by at least at least about 10%, at least about 20%, at least about 25%, at least about 30%, at least about 40%, at least about 50%, or more than 50%, compared to the serum AST level before treatment.

In some cases, an effective amount of an inhibitory nucleic acid of the present disclosure is an amount that, when administered in one or more doses to a subject, produces a desired result relative to a healthy subject. For example, an effective dose may be one that, when administered to a subject having elevated plasma glucose and/or plasma insulin, achieves a desired reduction relative to that of a healthy subject by at least about 10%, at least about 20%, at least about 25%, at least about 30%, at least about 40%, at least about 50%, at least about 60%, at least about 70%, at least about 80%, or more than 80%.

In some cases, an effective amount of an inhibitory nucleic acid of the present disclosure is an amount that, when administered in one or more doses to a subject, reduces BMI to within a normal range, e.g., to within 18.5 kg/m² to about 24.9 kg/m². In some cases, an effective amount of an inhibitory nucleic acid of the present disclosure is an amount that, when administered in one or more doses to a subject, reduces BMI by at least 5%, at least 10%, or at least 15%, where the starting BMI was greater than 30 kg/m².

### Routes of Administration

Suitable routes of administration include oral, rectal, nasal, pulmonary, topical, subcutaneous, intramuscular, intraperitoneal, intravenous, intradermal, intrathecal, and epidural. In some cases, the route of administration is intramuscular. In some cases, the route of administration is intravenous.

### Combination Therapy

The present disclosure contemplates the use of an inhibitory nucleic acid of the present disclosure in combination with one or more additional agents (e.g., one or more additional active therapeutic agents) or other prophylactic or therapeutic modalities. In such combination therapy, the various active agents frequently have different mechanisms of action. Such combination therapy may be especially advantageous by allowing a dose reduction of one or more of the agents, thereby reducing or eliminating the adverse effects associated with one or more of the agents; furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain cases, an inhibitory nucleic acid of the present disclosure and the at least one additional agent are administered or applied sequentially, e.g., where one agent is administered prior to one or more other agents. In other cases, an inhibitory nucleic acid of the present disclosure and the at least one additional agent are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

An inhibitory nucleic acid of the present disclosure can be used in combination with other agents useful in the treatment of the disorders or conditions set forth herein, including those that are normally administered to subjects suffering from obesity, eating disorder, hyperglycemia, hyperinsulinemia, glucose intolerance, and other glucose metabolism disorders.

The present disclosure contemplates combination therapy with numerous agents (and classes thereof), including 1) insulin, insulin mimetics and agents that entail stimulation of insulin secretion, including sulfonylureas (e.g., chlorpropamide, tolazamide, acetohexamide, tolbutamide, glyburide, glimepiride, glipizide) and meglitinides (e.g., mitiglinide, repaglinide and nateglinide); 2) biguanides (e.g., metformin, and its pharmaceutically acceptable salts, in particular, metformin hydrochloride, and extended-release formulations thereof, such as Glumetza™, Fortamet™, and Glucophage XR™) and other agents that act by promoting glucose utilization, reducing hepatic glucose production and/or diminishing intestinal glucose output; 3) alpha-glucosidase inhibitors (e.g., acarbose, voglibose and miglitol) and other agents that slow down carbohydrate digestion and consequently absorption from the gut and reduce postprandial hyperglycemia; 4) thiazolidinediones (e.g., rosiglitazone, troglitazone, pioglitazone, glipizide, balaglitazone, rivoglitazone, netoglitazone, AMG 131, MBX2044, mitoglitazone, lobeglitazone, IDR-105, troglitazone, englitazone, ciglitazone, adaglitazone, darglitazone that enhance insulin action (e.g., by insulin sensitization) including insulin, and insulin mimetics (e.g., insulin degludec, insulin glargine, insulin lispro, insulin detemir, insulin glulisine and inhalable formulations of each), thus promoting glucose utilization in peripheral tissues; 5) glucagon-like-peptides including DPP-IV inhibitors (e.g., alogliptin, omarigliptin, linagliptin, vildagliptin and sitagliptin) and Glucagon-Like Peptide-1 (GLP-1) and GLP-1 agonists and analogs (e.g., exenatide (BYETTA and ITCA 650 (an osmotic pump inserted subcutaneously that delivers an exenatide analog over a 12-month period; Intarcia, Boston, Mass.)) and GLP-1 receptor agonists (e.g., dulaglutide, semaglutide, albiglutide, exenatide, liraglutide, lixisenatide, taspoglutide, CJC-1131, and BIM-51077, including intranasal, transdermal, and once-weekly formulations thereof); and 6) and DPP-IV-resistant analogues (incretin mimetics), PPAR gamma agonists, PPAR alpha agonists such as fenofibric acid derivatives (e.g., gemfibrozil, clofibrate, ciprofibrate, fenofibrate, bezafibrate), dual-acting PPAR agonists (e.g., ZYH2, ZYH1, GFT505, chiglitazar, muraglitazar, aleglitazar, sodelglitazar, and naveglitazar), pan-acting PPAR agonists, PTPIB inhibitors (e.g., ISIS-113715 and TTP814), SGLT inhibitors (e.g., ASP1941, SGLT-3, empagliflozin, dapagliflozin, canagliflozin, BI-10773, PF-04971729, remogloflozin, TS-071, tofogliflozin, ipragliflozin, and LX-4211), insulin secretagogues, angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan, valsartan, candesartan, olmesartan, telmesartan); and the like.

### Subjects

Subjects suitable for treatment with a method of the present disclosure include individuals having a metabolic disorder. Subjects suitable for treatment with a method of the present disclosure include obese individuals. Subjects suitable for treatment with a method of the present disclosure include individuals having type 2 diabetes. Subjects suitable for treatment with a method of the present disclosure include individuals having diabetic retinopathy. Subjects suitable for treatment with a method of the present disclosure include individuals having non-alcoholic fatty liver disease (NAFLD). Subjects suitable for treatment with a method of the present disclosure include individuals having non-alcoholic steatohepatitis (NASH).

### Methods of Treating Muscular Dystrophy

The present disclosure provides methods of treating a muscular dystrophy disorder. The methods comprise administering to an individual in need thereof an effective amount of an inhibitory nucleic acid of the present disclosure, or a composition (e.g., a pharmaceutical composition) comprising same. In some cases, an inhibitory nucleic acid of the present disclosure is administered in a lipid nanoparticle. In some cases, an inhibitory nucleic acid of the present disclosure is administered in a liposome.

Muscular dystrophy disorders that can be treated with a method of the present disclosure include, e.g., Duchenne Muscular Dystrophy (DMD), Becker muscular dystrophy, myotonic muscular dystrophy, Facioscapulohumeral muscular dystrophy, and limb-girdle muscular dystrophy.

In some cases, an effective amount of an inhibitory nucleic acid of the present disclosure is an amount that, when administered in one or more doses to a subject, results in one or both of: i) improved overall metabolic profile, including mitochondrial homeostasis; and ii) reduced oxidative stress.

In some cases, an effective amount of an inhibitory nucleic acid of the present disclosure is an amount that, when administered in one or more doses to a subject, provides for an increase in muscle strength in the subject. For example, in some cases, an effective amount of an inhibitory nucleic acid of the present disclosure is an amount that, when administered in one or more doses to a subject, provides for an increase of at least 10%, at least 25%, at least 50%, at least 2-fold, or more than 2-fold, in muscle strength in the subject, compared to the level of muscle strength in the subject before being treated with the inhibitory nucleic acid.

In some cases, an effective amount of an inhibitory nucleic acid of the present disclosure is an amount that, when administered in one or more doses to a subject, provides for a reduction in skeletal muscle fibrosis and necrosis. For example, in some cases, an effective amount of an inhibitory nucleic acid of the present disclosure is an amount that, when administered in one or more doses to a subject, provides for a reduction of at least 10%, at least 25%, at least 50%, at least 60%, at least 70%, or more than 70%, in skeletal muscle fibrosis and necrosis, compared to the level of skeletal muscle fibrosis and necrosis in the subject before being treated with the inhibitory nucleic acid.

### Routes of Administration

Suitable routes of administration include oral, rectal, nasal, pulmonary, topical, subcutaneous, intramuscular, intraperitoneal, intravenous, intradermal, intrathecal, and epidural. In some cases, the route of administration is intramuscular. In some cases, the route of administration is intravenous.

### Subjects Suitable for Treatment

Subjects suitable for treatment using a method of the present disclosure include individuals having DMD, Becker muscular dystrophy, myotonic muscular dystrophy, Facioscapulohumeral muscular dystrophy, or limb-girdle muscular dystrophy.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An inhibitory nucleic acid comprising the nucleotide sequence:

a)
(SEQ ID NO: 1)
5′-ACCGGTTCACTGTG-3′;
or
b)
(SEQ ID NO: 2)
5′-GACCGGTTCACTGT-3′;
or
c)
(SEQ ID NO: 3)
5′-AGACCGGTTCACTGTG-3′,

wherein the inhibitory nucleic acid comprises one or more locked nucleic acids (LNA).

Aspect 2. The inhibitory nucleic acid of aspect 1, wherein the inhibitory nucleic acid comprises: i) a modified backbone; and/or ii) one or more 5-methyl deoxycytosine residues.

Aspect 3. The inhibitory nucleic acid of aspect 1 or aspect 2, wherein the inhibitory nucleic acid has a length of from 14 nucleotides to 20 nucleotides.

Aspect 4. The inhibitory nucleic acid of any one of aspects 1-3, wherein the inhibitory nucleic acid comprises the nucleotide sequence (including the modifications) depicted in FIG. 1A and designated “NRC0090.”

Aspect 5. The inhibitory nucleic acid of any one of aspects 1-3, wherein the inhibitory nucleic acid comprises the nucleotide sequence (including the modifications) depicted in FIG. 1A and designated “NRC-0091.”

Aspect 6. The inhibitory nucleic acid of any one of aspects 1-3, wherein the inhibitory nucleic acid comprises the nucleotide sequence (including the modifications) depicted in FIG. 1A and designated “NRC-0119.”

Aspect 7. A pharmaceutical composition comprising:
a) an inhibitory nucleic acid of any one of aspects 1-6; and
b) a pharmaceutically acceptable excipient.

Aspect 8. The pharmaceutical composition of aspect 7, wherein the pharmaceutically acceptable excipient comprises one or more lipids.

Aspect 9. The pharmaceutical composition of aspect 7, wherein the pharmaceutically acceptable excipient comprises poly(amidoamine), poly(propyleneimine), or poly(L-lysine).

Aspect 10. A lipid nanoparticle comprising: a) an inhibitory nucleic acid of any one of aspects 1-6; and b) a pharmaceutically acceptable excipient.

Aspect 11. A method of treatment, the method comprising administering to an individual in need thereof an effective amount of an inhibitory nucleic acid of any one of aspects 1-6, a pharmaceutical composition of any one of aspects 7-9, or a lipid nanoparticle of aspect 10.

Aspect 12. A method of treating a metabolic disorder in an individual, the method comprising administering to the individual an effective amount of an inhibitory nucleic acid of any one of aspects 1-6, a pharmaceutical composition of any one of aspects 7-9, or a lipid nanoparticle of aspect 10.

Aspect 13. The method of aspect 12, wherein the metabolic disorder is insulin resistance, hyperglycemia, type 2 diabetes mellitus, obesity, fatty liver disease, glucose intolerance, hyperinsulinemia, metabolic syndrome, or hypertension.

Aspect 14. The method of aspect 12, wherein the metabolic disorder comprises insulin resistance.

Aspect 15. The method of aspect 12, wherein the metabolic disorder comprises metabolic syndrome.

Aspect 16. The method of aspect 12, wherein the metabolic disorder comprises type 2 diabetes mellitus.

Aspect 17. The method of any one of aspects 12-16, wherein the individual has a body mass index>30.0.

Aspect 18. The method of any one of aspects 12-17, wherein said administering results in a serum insulin level in a normal range.

Aspect 19. The method of any one of aspects 12-17, wherein said administering results in a blood glucose level in a normal range.

Aspect 20. The method of any one of aspects 12-19, further comprising administering at least one additional therapeutic agent.

Aspect 21. The method of aspect 20, wherein the at least one additional therapeutic agent is insulin, an insulin analog, a biguanidine, or a thiazolidinedione.

Aspect 22. The method of any one of aspects 11-20, wherein said administering is via oral administration.

Aspect 23. A method of treating muscular dystrophy in an individual, the method comprising administering to the individual an effective amount of an inhibitory nucleic acid of any one of aspects 1-6, a pharmaceutical composition of any one of aspects 7-9, or a lipid nanoparticle of aspect 10.

Aspect 24. The method of aspect 23, wherein the muscular dystrophy is Duchenne muscular dystrophy or Becker muscular dystrophy.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

FIG. 1A depicts sequence information for three ASOs. ID indicates drug name, OLIGO indicates nucleotide sequence (uppercase denotes locked nucleic acids (LNAs), lowercase denotes standard bases), STRING indicates modifications (+ sign precedes an LNA, * precedes a phosphorothioated base, /iMe-dC/ denotes an internal 5-methyl deoxycytosine, Length indicates number of nucleotides, #LNAs indicates number of standard bases that have been replaced with LNAs. FIG. 1B provides a schematic LNA/DNA composition of each ASO and their base-pairing location within the target miRNA (miR-128-3p). The seed sequence of miR-128-3p is denoted in red.

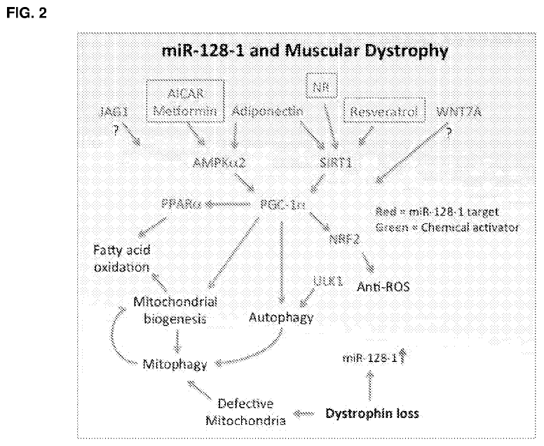

In this disclosure, it is shown that miR-128-1 miRNA represents an intriguing therapeutic target in DMD due to its regulation of skeletal muscle mitochondrial metabolism and direct targeting of several key modifiers downstream of dystrophin loss (FIG. 2). To investigate the therapeutic potential and action mechanism of miR-128-1 in DMD, locked nucleic acid antisense oligonucleotide (LNA ASOs) targeting miR-128-1 were developed. A highly efficient depletion of miR-128-1 in skeletal muscle was achieved via once-weekly subcutaneous injection. Furthermore, miR-128-1 was identified as implicated in regulating a number of the genes shown to act as modifiers of DMD phenotypes, including PGC-1α, AMPKα2, SIRT1, JAG1 and Wnt7A, as well as other metabolic regulators such as PPARα, PPARγ, ULK1, CPT1β, and others.

FIG. 2. Dystrophin loss in DMD muscle cells results in abnormal mitochondria function, and a number of regulators of mitochondrial biology and autophagy, as well as certain developmental regulators such as Notch and Wnt signaling components can at least partially rescue DMD pathologies. Interestingly, many of these regulators are predicted or verified targets of the miR-128-1 miRNA.

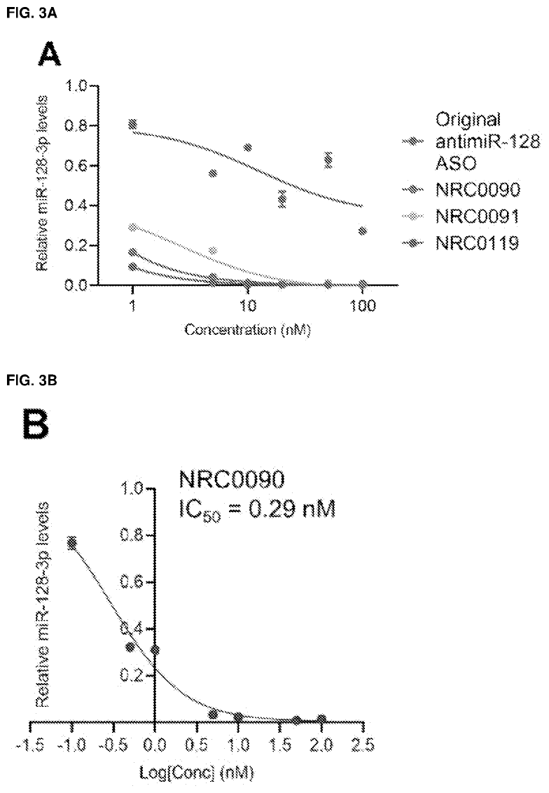

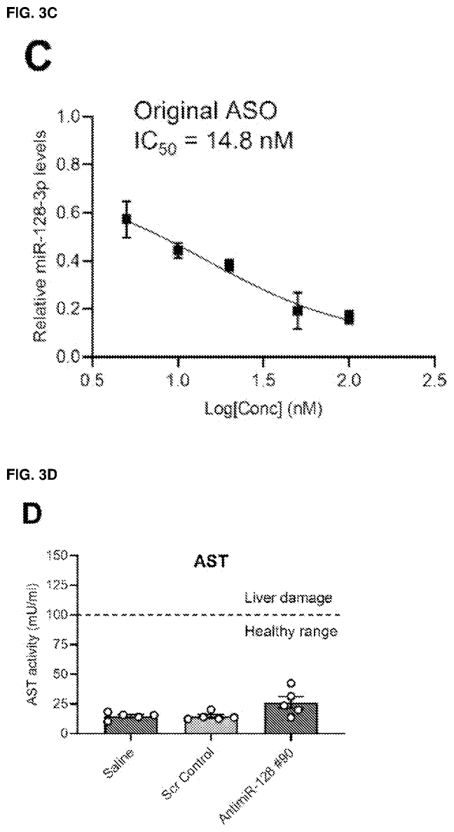

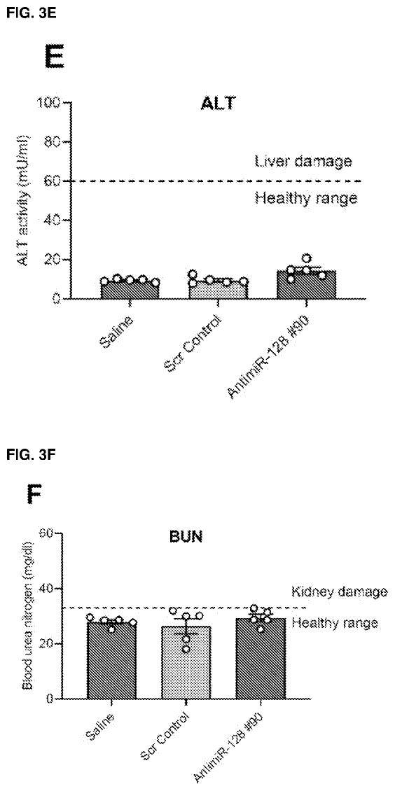

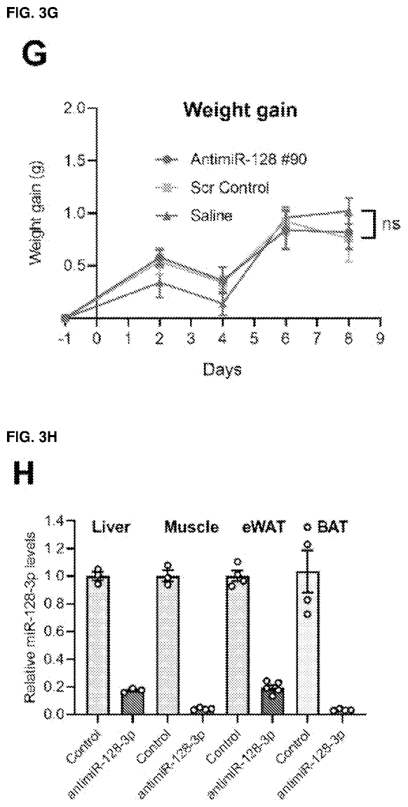

A new generation of anti-miR-128-3p ASOs with vastly increased potency has been developed. The ASO design algorithm alters sequence, length, and LNA position to maximize binding potency and accessibility to the miR-128-3p seed region. Melting temperature and binding energy are also optimized through this process, while self-complementarity and off-target binding are minimized. NRC0090, NRC0091, and NRC0119 scored highest in the stringent screening process, in vitro testing reveals that these ASOs are 20- to 50-fold more potent than the previous antimiR-128-3p ASO prototype^{1,2} (FIG. 3). Importantly, the new ASO appears well tolerated and non-toxic when administered at high doses to mice (FIG. 3), and short-term treatment to mice results in successful and potent inhibition of miR-128-3p levels in liver, muscle, eWAT, and BAT tissues (FIG. 2).

FIG. 3. New miR-128-3p-targeting LNA ASOs are up to 50-fold more potent than the original LNA ASO prototype, and demonstrates on-target in vivo efficacy without toxicity. (A-C) Dose-response curves for candidate and originally reported ASOs. Huh-7 hepatoma cells were transfected with antimiR-128-3p LNA ASO or scrambled control and assayed for miR-128-3p levels via RT-qPCR after (A) 72 h, or (B-C) 24 h. miR-128-3p in treatment groups was normalized to U6 reference RNA, then to the scrambled control. (D-G) In vivo LNA ASO tolerability. 9 week-old male C57BL/6 mice (n=5) were s.c. injected with repeated 20 mg/kg doses of NRC0090, scrambled control ASO, or saline on days 0, 2, 4, and 6. Body weight gain was monitored and a terminal blood draw was performed on day 8 for measurement of liver (AST/ALT) and kidney (BUN) toxicity markers. (H) In vivo antimiR-128-3p LNA ASO targeting study. 7 week-old male C57BL/6 mice (n=5) were placed on a high-fat diet and s.c. injected with 10 mg/kg of antimiR-128-3p LNA ASO or scrambled control. Treatments were administered on day 0 and day 7, and indicated tissues were harvested on day 10 for miR-128-3p quantification by RT-qPCR. miR-128-3p in treatment groups was normalized to U6 reference RNA, then to the scrambled control. Data are presented as mean and SEM.

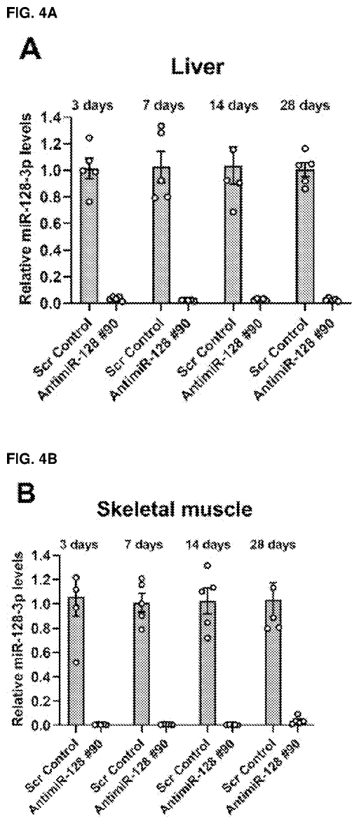

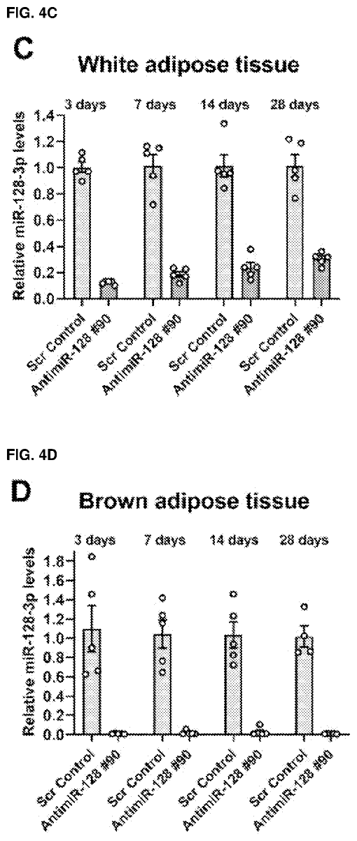

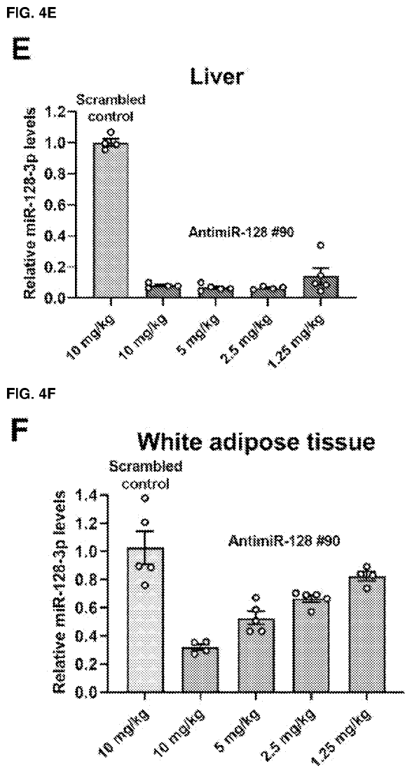

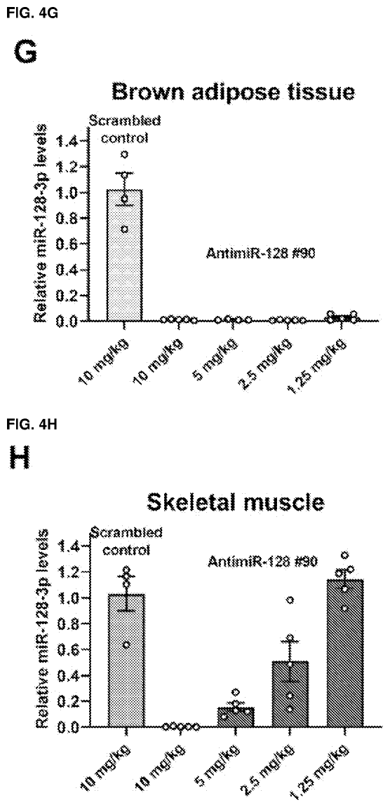

The LNA chemistry affords strongly increased target affinity and specificity, and is combined with a phosphorothioate backbone of the ASO for increased in vivo stability and pharmacokinetics. Terminal LNA ASO half-life in circulation is typically 2-3 weeks in mammals, based on multiple studies in mice and non-human primates with an LNA ASO targeting miR-33¹⁸, with LNA ASOs detected in tissues up to 7 weeks after a single injection. Dosing studies in mice show that target inhibition is maintained 28 days after a single injection, allowing for once-weekly and possibly once-monthly dosing (FIG. 4). Furthermore, dose response studies show strong target engagement in all major metabolic tissues, including skeletal muscle, liver, subcutaneous and visceral WAT, and BAT (FIG. 4).

FIG. 4. Dose optimization studies in mice. (A-D) Time course study. 8 week-old C57BL/6 mice (n=5) on a standard diet were injected s.c. with 10 mg/kg NRC0090 antimiR-128-3p or scrambled control ASO on day 0. Tissues were harvested from cohorts on days 3, 7, 14, and 28 for miR-128-3p quantification by RT-qPCR. miR-128-3p in treatment groups was normalized to U6 reference RNA, then to the scrambled control. Data are presented as mean and SEM. (E-I) Dose response study in mice. 7 week-old C57BL/6 mice (n=5) were placed on a high-fat diet and injected s.c. with indicated dose of NRC0090 antimiR-128-3p or scrambled control ASO on day 0. Tissues were harvested on day 28 for miR-128-3p quantification by RT-qPCR. miR-128-3p in treatment groups was normalized to U6 reference RNA, then to the scrambled control. Data are presented as mean and SEM.

### Example 2: Metabolic Syndrome

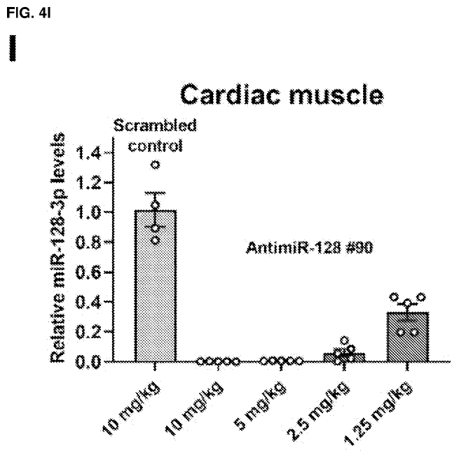

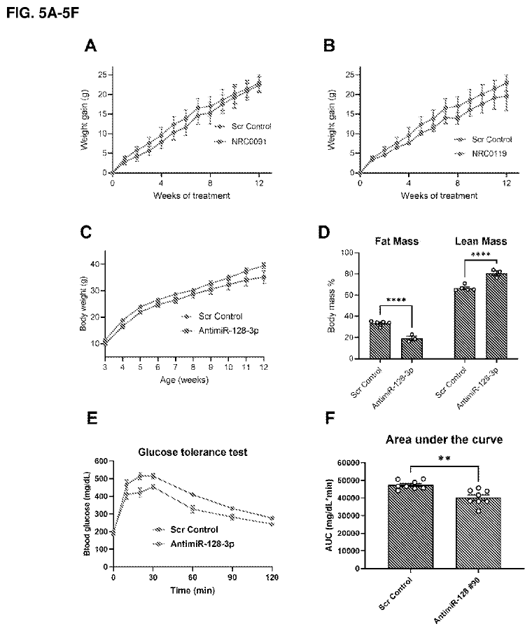

NRC0090, NRC0091, and NRC0119 have been tested in diet-induced obese mice and shown to have protective effects against conditions associated with MetS. These include body weight gain, fat mass accumulation, and glucose intolerance (FIG. 5).

FIG. 5. (A-B) 7 week-old male C57BL/6 mice (n=3) were placed on a 60% high-fat diet and s.c. injected with 10 mg/kg/week of indicated antimiR-128-3p LNA ASO or scrambled control and body weights were monitored with time. (C-D) 3 week-old male C57BL/6 mice (n=5) were placed on a 60% high-fat diet and s.c. injected with 10 mg/kg/week of NRC0090 antimiR-128-3p LNA ASO or scrambled control. Body weights were monitored weekly (C), and fat and lean mass were measured via EchoMRI at the study termination (D). (E-F) 6 week-old male C57BL/6 mice (n=10) were placed in a 60% high-fat diet and s.c. injected with 5 mg/kg/month of NRC0090 antimiR-128-3p LNA ASO or scrambled control. After 15 weeks of treatment, mice were fasted 6 hrs, then i.p. injected with 1 g/kg D-glucose, and blood glucose levels were monitored with time (E). Area under the curve was calculated using GraphPad employing a Y=0 baseline, and data were analyzed via one-way ANOVA (F).

### Example 3: Duchenne Muscular Dystrophy

Duchenne Muscular Dystrophy (DMD) is caused by mutations in the dystrophin gene, resulting in progressive muscle degeneration and weakness and ultimately premature death due to respiratory and cardiac failure. To date, there is no cure for DMD. Steroids aim only to control the symptoms and come with many serious adverse effects. Newly developed approaches focused on restoring partially functional dystrophin (e.g., exon skipping antisense oligonucleotides (ASOs) and AAV-micro-Dystrophin) show limited efficacy for a relatively small fraction of patients, and the duration of effects is uncertain. Thus, additional efforts have been made to mitigate the disease by ameliorating the deleterious effects downstream of dystrophin loss, which include metabolic abnormalities associated with mitochondrial dysfunction, elevated reactive oxygen species (ROS), fatty infiltration, inflammation, and fibrosis.

miRNAs are short (18-24 nucleotides) regulatory non-coding RNAs that function in development, metabolism, and disease by epigenetically regulating specific target genes through their 3′ untranslated regions (3′UTRs). A number of miRNAs have been shown to be upregulated in DMD muscle, so called “dystro-miRs”; however, convincing data showing that therapeutic targeting of dystro-miRs ameliorate DMD phenotypes in animal models is lacking.

The mature miR-128-3p miRNA (derived from the miR-128-1 locus on human chromosome 2 and miR-128-2 on chromosome 3; also known as miR-128a and miR-128b, respectively) has been identified as a master regulator of metabolic homeostasis. Particularly, miR-128-3p expression negatively regulates mitochondrial respiration and biogenesis and suppresses expression of key genes involved in mitochondrial function and homeostasis.

To block miR-128-3p action, state-of-the-art locked nucleic acid antisense oligonucleotides (LNA ASOs) were engineered. The miR-128-3p LNA ASO has been modified to enable subcutaneous delivery and robustly targets miR-128-3p in the skeletal and cardiac muscle. In this study, the efficacy of the anti-miR-128-3p LNA ASO as a treatment in the mdx^{5cv} mouse and DMD^{Y/-} pig models was examined. Near complete rescue of skeletal muscle and cardiac dysfunction was demonstrated in these models, respectively. Furthermore, the well-established metabolic perturbations associated with DMD, including glucose intolerance, mitochondrial dysfunction, and oxidative stress, are markedly improved to near wild-type levels with treatment.

In a mouse model of Duchenne muscular dystrophy (mdx^{5cv}), it was discovered that anti-miR-128-1 treatment dramatically improved muscle strength and endurance, accompanied by reduced skeletal muscle fibrosis and necrosis, with an excellent overall safety profile. This therapeutic effect was through improved overall metabolic profile, including mitochondrial homeostasis, reduced oxidative stress, and lipid oxidation, etc. Interestingly, the effect of the ASO on the small intestine of mdx^{5cv} mice is also demonstrated.

Elevated miR-128-3p Expression is Linked to Muscle Weakness in Human DMD Patients and in DMD Animal Models

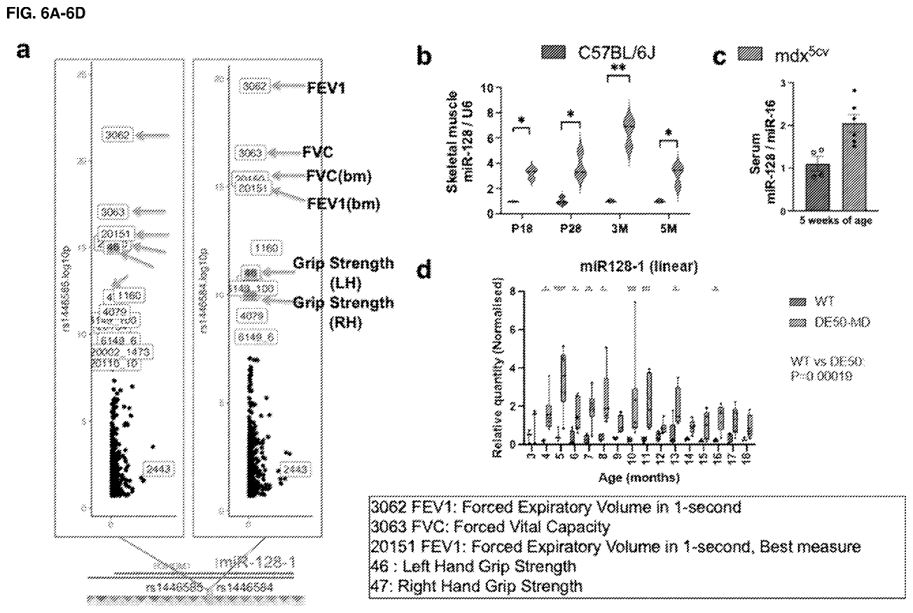

Using the UK biobank (n>300,000) human data, the genetic link between the miR-128-3p-encoding miR-128-1 and miR-128-2 loci and human grip strength and respiratory function was found (FIG. 6A). The expression of miR-128-3p across DMD animal models and this data indicates that miR-128-3p is elevated in the mdx^{5cv} mouse model (FIG. 6B, C) as well as in the DE50-MD dog model (FIG. 6D). This dovetails with the literature on DMD patients, which shows that miR-128-3p is elevated in patient plasma and skeletal muscle tissues.

FIG. 6. miR-128-3p expression is linked to muscle weakness in humans and DMD animal models. (A) The miR-128-3p locus is genetically linked to weak hand grip strength and pulmonary weakness (UK Biobank; n>300,000), and miR-128-3p is elevated in DMD mouse and dog models. The miR-128-3p genomic locus on human chromosome 2 is linked to weak left/right hand grip strength and impaired lung muscle function (forced expiratory volume and forced vital capacity (B,C) Skeletal muscle and serum miR-128-3p level was measured in WT and mdx^{5cv} mice at various ages. n=5 per group. (D) Level of miR-128-3p was measured in DMD dog model DE50-MDI from 3 months of age to 18 months of age.

LNA ASO Treatment Targeting miR-128-3p in the Mdx^{5cv} DMD Mouse Model Ameliorates all Pathological Phenotypes

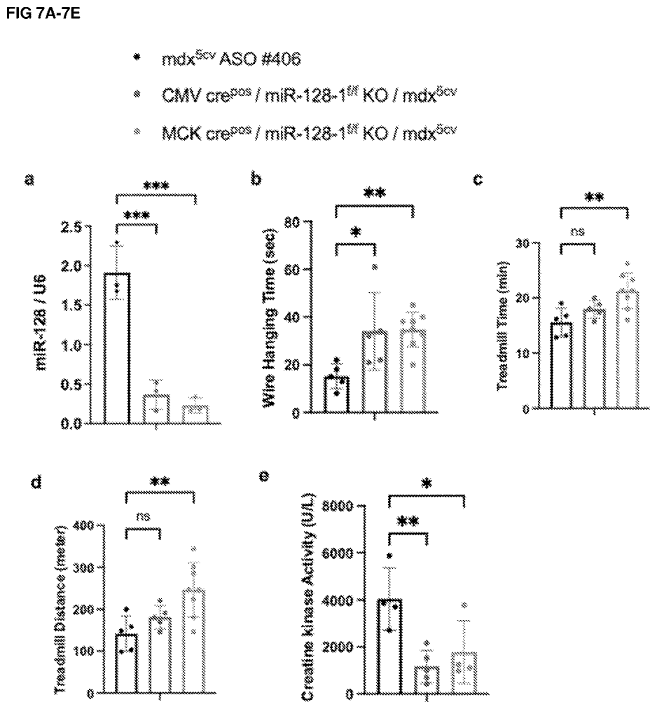

To assess whether miR-128-3p is a pathological modifier in DMD, mdx^{5cv} mice were crossed with whole body miR-128-3p knockout mice or muscle-specific miR-128-3p knockout mice. It was hypothesized that knockdown of miR-128-3p expression would improve skeletal muscle function in mdx^{5cv} mice. These results indicate that miR-128-3p expression was downregulated to 36.6% and 22.9%, respectively, in whole body miR-128-3p knockout mice or muscle-specific miR-128-3p knockout mice when compared to wild-type mice (FIG. 7A). Muscle grip strength was significantly increased in both mouse models compared to mdx^{5cv} mice (FIG. 7B). Exercise endurance was tested with treadmill running until exhaustion, and the data indicate that knockout of miR-128-3p in skeletal muscle significantly improved endurance (FIG. 7C, D), as well as reduced circulating creatine kinase levels (a muscle damage marker; FIG. 7E). Taken together, this shows that miR-128-3p expression contributes to DMD pathology in mdx^{5cv} mice.

FIG. 7. Whole body or muscle specific miR-128-3p knockout improves muscle function in mdx^{5cv} mice. (A) miR-128-3p levels were measured in the GA muscle. (B) Passive wire hanging for muscle grip strength was measured. (C, D) Treadmill running until exhaustion was used to measure exercise endurance. Time and distance were plotted respectively. (E) Creatine kinase in serum was measured to examine muscle damage.

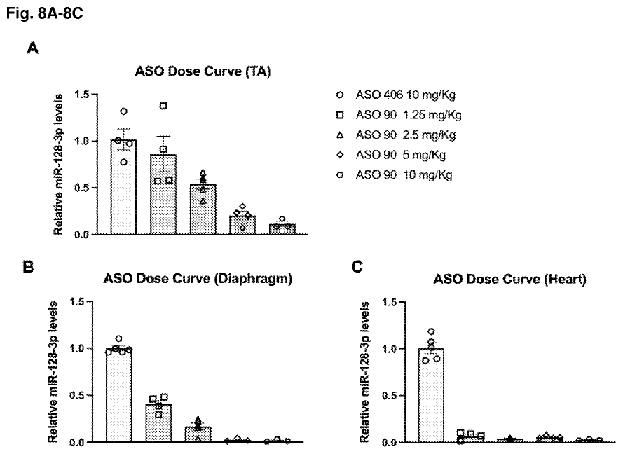

To determine whether the use of LNA ASOs to suppress miR-128-3p can be applied as a therapy for DMD, state-of-the-art LNA ASOs capable of targeting miR-128-3p and robustly downregulating its expression were generated. First, a dose response study was performed to evaluate the knockdown efficiency of this LNA ASO in mdx^{5cv} mouse tissues. Expression levels of miR-128-3p were measured in skeletal muscle, diaphragm, and cardiac muscle seven days after a single subcutaneous injection at a series of doses. This data demonstrated dramatic inhibition of miR-128-3p in all of these tissues (FIG. 8). Based on this data, a dose of 10 mg/kg delivered once-weekly subcutaneously was selected for subsequent mouse studies.

FIG. 8. In vivo ASO dose response in mdx^{5cv} mice. Mice were injected with control ASO 406 at 10 mg/kg or anti-miR-128-3p ASO 90 at a range of doses from 1.25 mg/kg to 10 mg/kg. TA, Diaphragm and heart were harvested 7 days after one injection. (A-C) miR-128-3p expression level in the TA (A), diaphragm (B), and heart (C). Data are normalized to the control ASO 406 group.

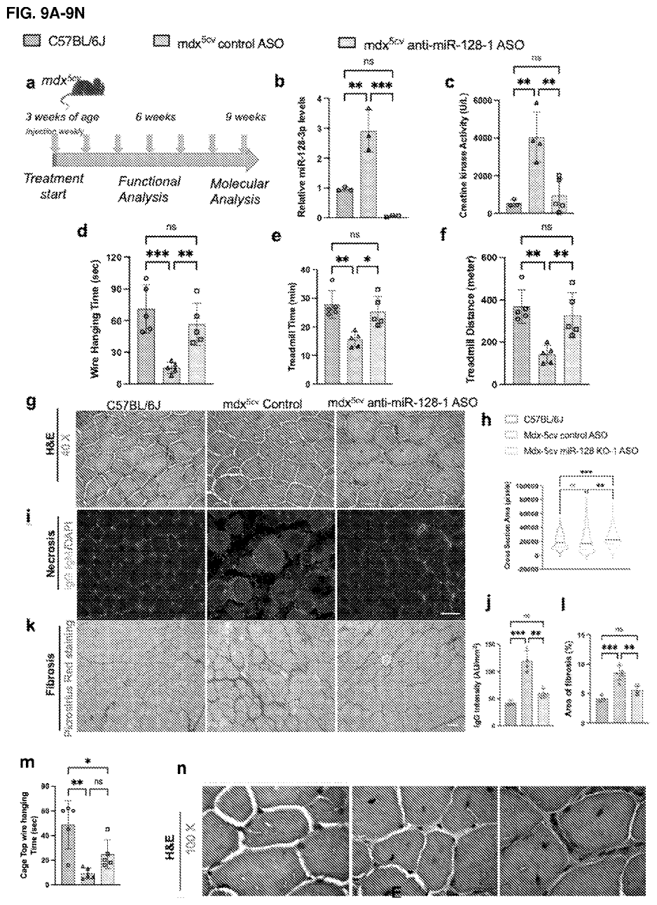

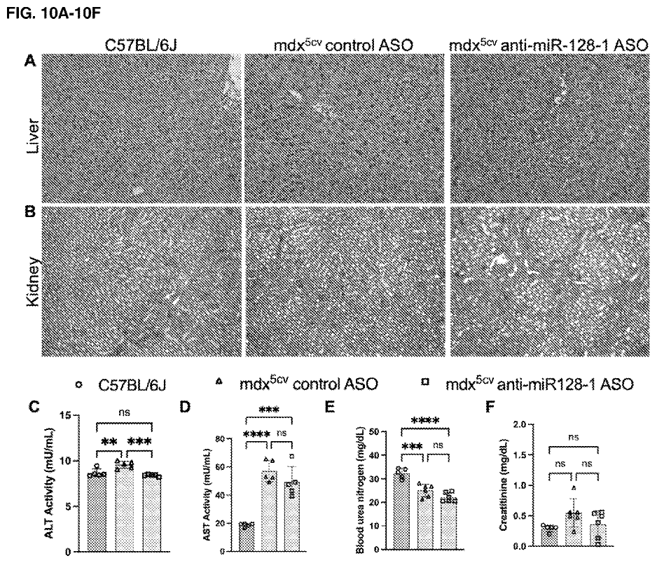

Considering the early onset of DMD, anti-miR-128-3p LNA ASO treatment was started at 3 weeks of age and continued until 6 or 9 weeks of age in mdx^{5cv} mice (FIG. 9A). Analysis of miR-128-3p expression levels demonstrated robust (>95%) knockdown sustained over the treatment period (FIG. 9B). miR-128-3p knockdown markedly ameliorated many pathological manifestations of dystrophin loss. First, this data demonstrated a dramatic decrease in circulating creatine kinase (FIG. 9C), significant rescue of the muscle grip strength (FIG. 9D), and markedly improved endurance exercise capacity (FIG. 9E, F). Muscle histology revealed an increase in muscle fiber diameters, decreased muscle fiber necrosis, and a reduction in muscle fibrosis (FIG. 9G-L). Furthermore, the anti-miR-128-3p LNA ASO is well tolerated in these mice and shows no apparent liver and kidney toxicity (FIG. 10A-10F).

FIG. 9. Anti-miR-128-3p LNA ASO ameliorated pathological phenotypes of the mdx^{5cv} DMD mouse model. (A) Schematic of experimental design. (B) miR-128-3p expression level in the GA muscle of WT and mdx^{5cv} mice with control ASO and treatment ASO. (C) Muscle damage marker circulating creatine kinase activity was measured. (D) Two-limb wire hanging for muscle grip strength was measured. (E, F) Treadmill running until exhaustion was used to measure exercise endurance. Time and distance were plotted respectively. (G) H&E images of TA muscle histology images at 40× and 100×. (H) Quantification of the TA muscle fiber size in all three experimental groups. (I) Necrosis was detected by staining with IgG antibody, and the fluorescence intensity was quantified as shown in (J). (K) TA muscle cryosections were stained with Picrosirius Red for fibrosis. (L) Quantification of the fibrotic area in three experimental groups. n=3-5 per group. (M) Two limb cage top wire hanging were measured in all three groups, respectively. (N) H&E images of TA muscle histology images at 100×.

FIG. 10. ASO injection is safe and non-toxic in the liver and kidney. (A, B) Liver and kidney H&E images from WT mice and mdx^{5cv} mice treated with control ASO and anti-miR-128-3p ASO. (C, D) Serum enzymatic levels of liver ALT and AST in WT and mdx^{5cv} mice treated with control ASO and anti-miR-128-3p ASO. (E, F) Serum blood urea nitrogen (BUN) and creatinine were measured as indicators for kidney function.

### Anti-miR-128-3p LNA ASO Treatment Ameliorates Metabolic Dysfunction in DMD

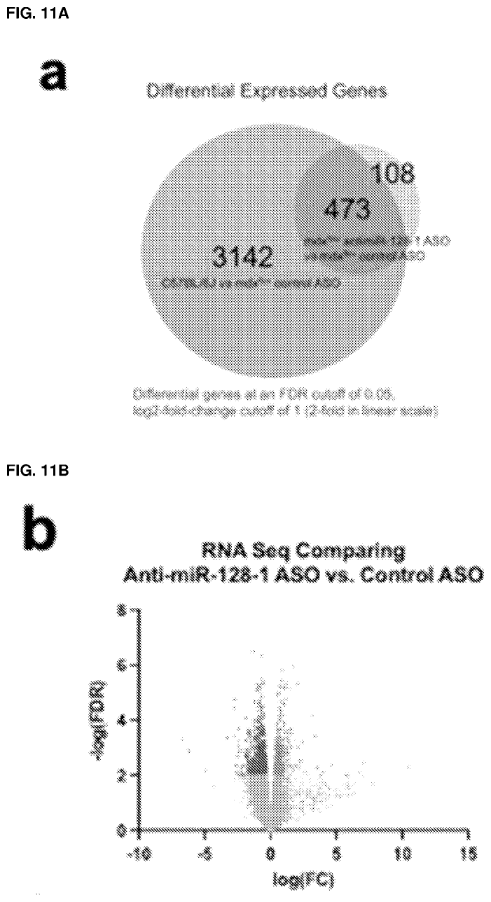

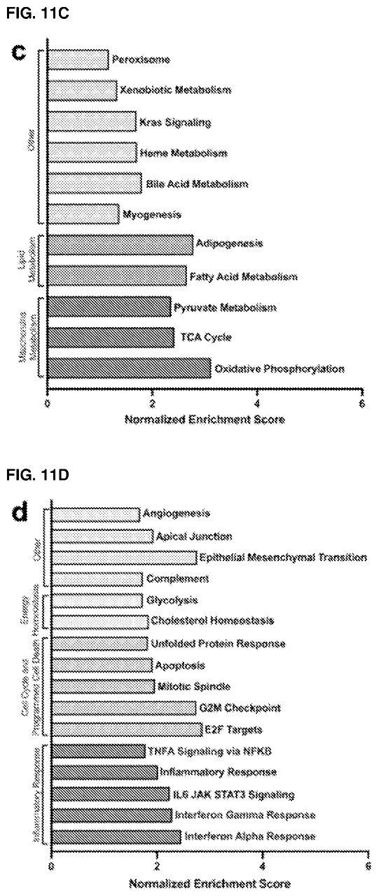

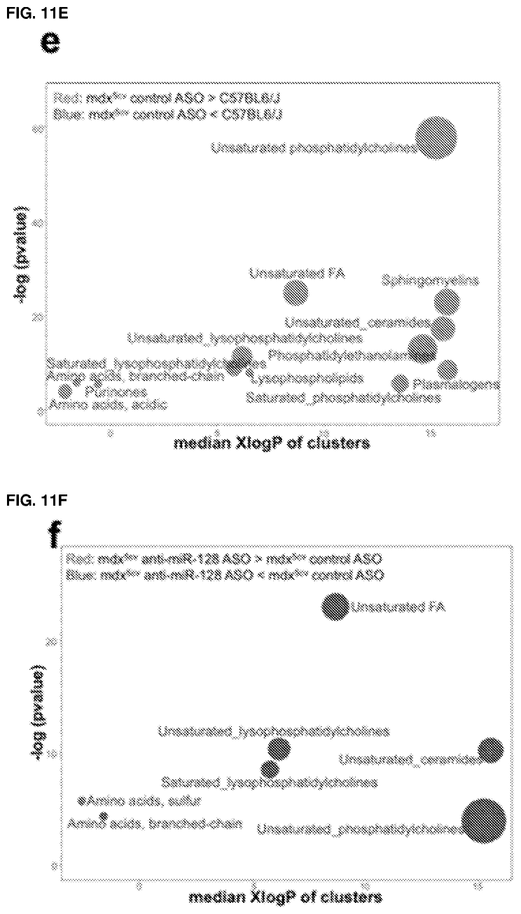

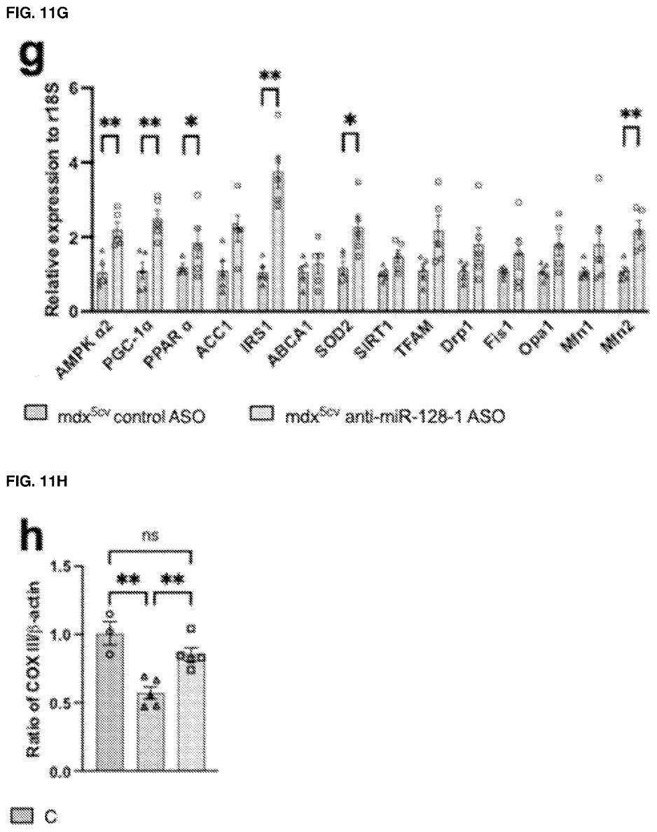

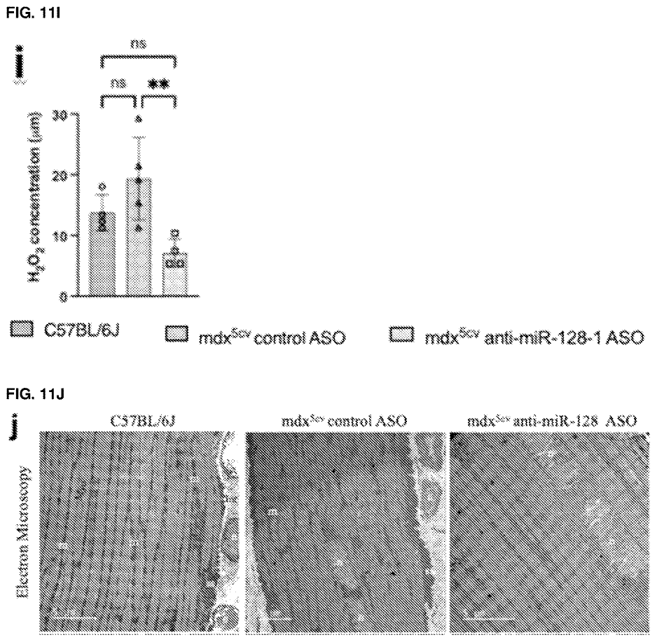

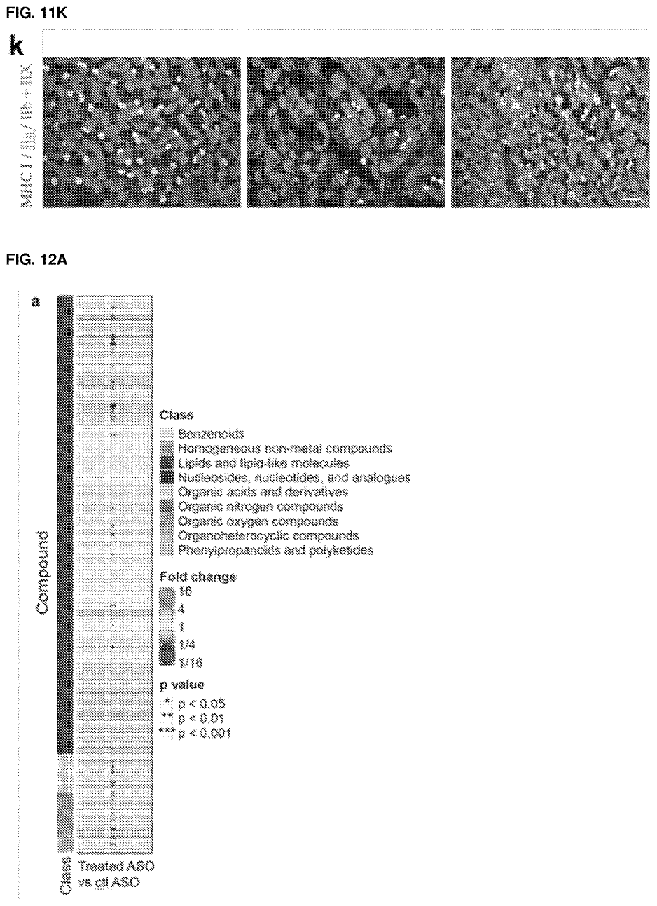

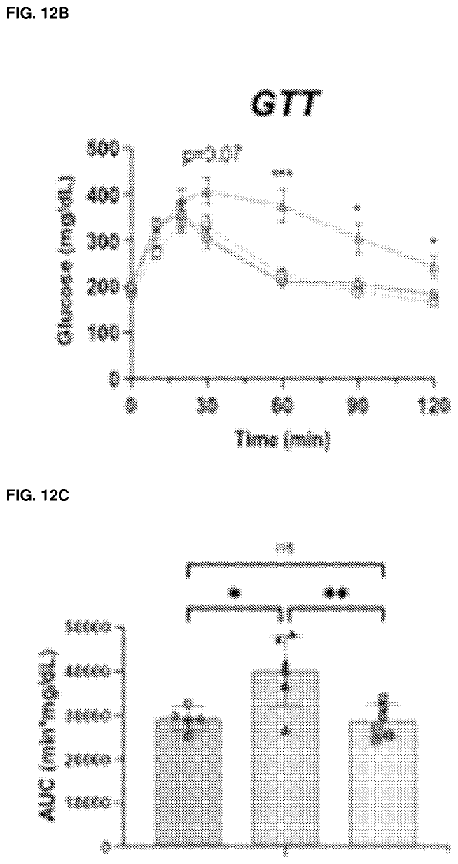

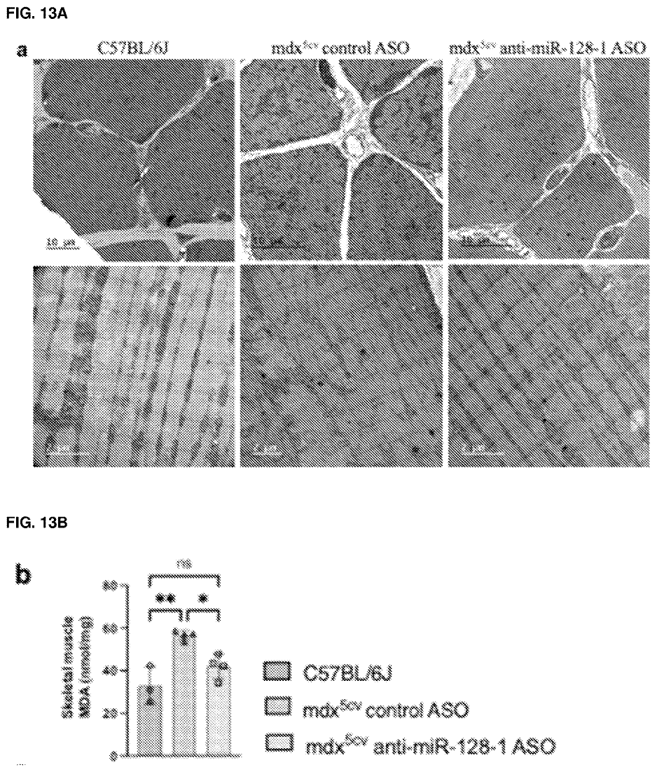

DMD patients and animal models exhibit marked metabolic dysfunction, such as glucose intolerance, reduced glycolytic and oxidative enzyme expression, impaired mitochondrial homeostasis and function, increased oxidative stress, and aberrant intrafibral and plasma lipid levels. In order to elucidate the mechanistic role of miR-128-3 in DMD pathology, RNA sequencing (RNA-seq) was performed on gastrocnemius muscle from mdx^{5cv} mice treated with anti-miR-128-3p or scramble control LNA ASO in comparison to wild-type mice. These studies revealed 3,142 differentially expressed genes in the mdx^{5cv} disease model compared with wild-type control, and the expression of 473 of these genes changed in response to anti-miR-128-3p treatment (FIG. 11A, B). The most significantly up-regulated pathways included oxidative phosphorylation, the TCA cycle, pyruvate metabolism, fatty acid metabolism, adipogenesis, and myogenesis (FIG. 11C). Significantly down-regulated pathways included interferon response, IL6-JAK-STAT3 signaling, and inflammatory response (FIG. 11D). The RNA-seq data highlights a number of changes in metabolic pathways. To examine the influence of anti-miR-128-3p LNA ASO treatment on metabolites in dystrophic muscle tissue, metabolomic analysis was performed. Several classes of metabolites were changed with LNA ASO treatment, with lipid species representing the most prominent category (FIG. 12A). Clustering of these metabolites demonstrates an increase in toxic lipid species in the dystrophic condition, which is ameliorated with LNA ASO treatment (FIG. 11E, F). These species include ceramides which, when elevated, are known to cause insulin resistance in skeletal muscle. To further explore this, an intraperitoneal glucose tolerance test was performed in mdx^{5cv} mice treated with anti-miR-128-3p LNA ASO. These results revealed a striking improvement in glucose clearance with LNA ASO treatment (FIG. 12B, C).

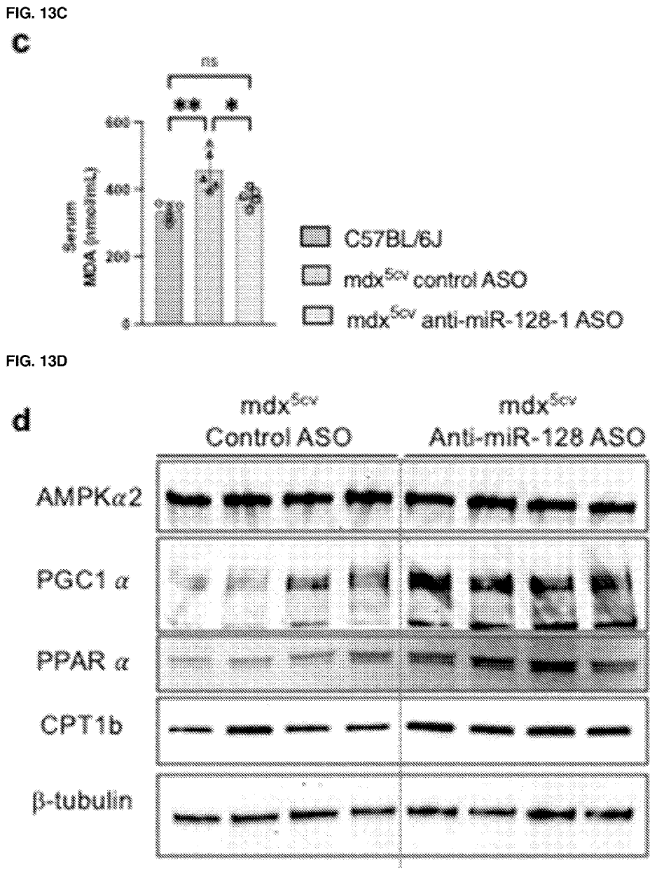

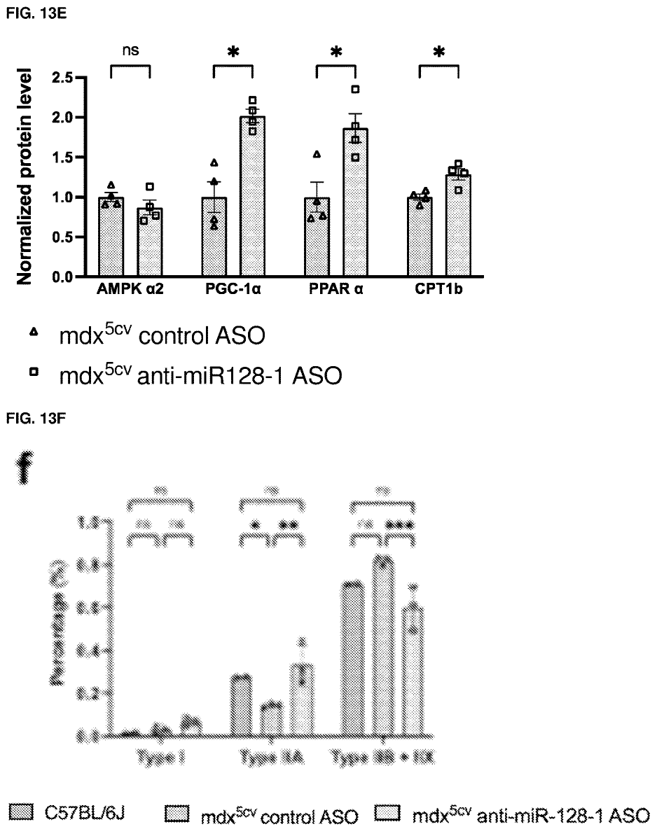

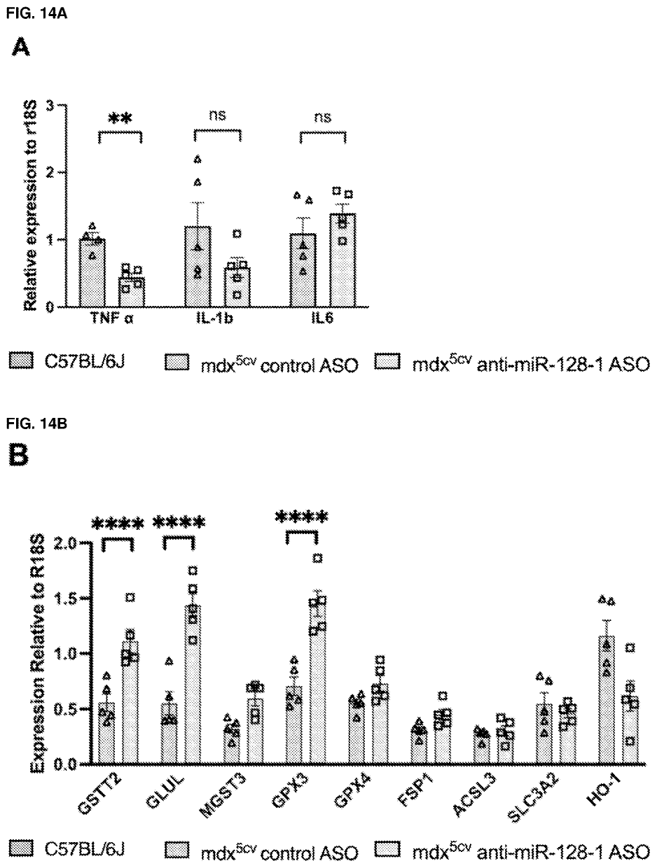

Mitochondrial dysfunction is also a well-established metabolic abnormality in DMD pathology. This RNA-seq analysis revealed that LNA ASO treatment increased expression of several mitochondria related pathways. To further investigate mitochondrial homeostasis, the expression of key regulators of mitochondrial function, biogenesis, and dynamics was examined. It was found that several of these regulators, including AMPKα2, PGC-1α, and PPARα, are significantly derepressed with anti-miR-128-3p LNA ASO treatment (FIG. 11G, FIG. 13D, E). Mitochondrial DNA copy number was significantly increased upon anti-miR-128-3p LNA ASO treatment suggesting an increase in mitochondrial biogenesis (FIG. 11H). Electron microscopy analysis of mitochondrial ultrastructure indicated improvement in aberrant mitochondrial integrity in mdx^{5cv} mice treated with anti-miR-128-3p LNA ASO (FIG. 11J). Specifically, mdx^{5cv} soleus muscle showed perinuclear mitochondrial localization which was resolved with anti-miR-128-3p LNA ASO treatment (FIG. 13A). Additionally, mitochondria are longitudinally mis-aligned in mdx^{5cv} soleus muscle, but correct mitochondrial alignment was recovered with anti-miR-128-3p LNA ASO treatment. In line with the finding of improved mitochondrial morphology, reduction in hydrogen peroxide levels and decreased lipid peroxidation was also observed with treatment indicating decreased oxidative stress in mdx^{5cv} mice treated with anti-miR-128-3p LNA ASO (FIG. 11I, FIG. 13B, C). Furthermore, muscle fiber composition shifted in favor of oxidative fiber types with an increase of type IIa fibers and a decrease of type IIb fibers (FIG. 11K, FIG. 13F). This fiber type switching is consistent with the observed de-repression of PGC-1α expression. Taken together, these data show an overall improvement in mitochondrial health.

FIG. 11. Anti-miR-128-3p LNA ASO treatment ameliorates metabolic dysfunction in DMD. (A-D), RNA-seq analysis of WT mice and mdx^{5cv} mice treated with control ASO and anti-miR-128-3p ASO. (A, B) Venn diagram and volcano plot presentations of the differentially expressed genes. (C, D) Enriched pathway analysis shows the upregulated (C) and downregulated (D) signaling cascades. (E, F) Analysis of metabolites changed in the gastrocnemius muscle of the mdx^{5cv} mouse model compared with wildtype (E) and mdx^{5cv} mice treated with control or anti-miR-128-3p LNA ASO (F). (G) De-repression of genes regulating mitochondrial homeostasis was measured by RT-qPCR. (H) Mitochondrial DNA copy number were measured in the soleus at 6 weeks of age. (I) Oxidative stress was quantified. (J) Longitudinal sections of soleus muscle by electron microscopy showed mitochondrial relocation and loss of organization at 9 weeks of age. (K) Three muscle fiber types were stained by immunofluorescence.

FIG. 12. Metabolomic analysis shows reduction of lipotoxic species upon miR-128-3p LNA ASO treatment of mdx^{5cv} mice which improves glucose tolerance. (A) Heat map of the fold change in metabolite species with miR-128-3p LNA ASO treatment in mdx^{5cv} mice. (B,C) Intraperitoneal glucose tolerance test was performed at six weeks of age. Area under the curve was quantified in (C).

FIG. 13. Anti-miR-128-3p LNA ASO treatment improves mitochondrial health in DMD. (A) Electron microscopy of soleus muscle was imaged from cross sections (top) and longitudinal sections (bottom). (B, C) Lipid peroxidation was measured in the skeletal muscle and serum indicated with MDA production. (D) Western blot was performed to measure mitochondrial protein levels in TA muscle. (E) Quantification of mitochondrial protein levels. (F) Muscle fiber type (shown in FIG. 3K) was quantified.

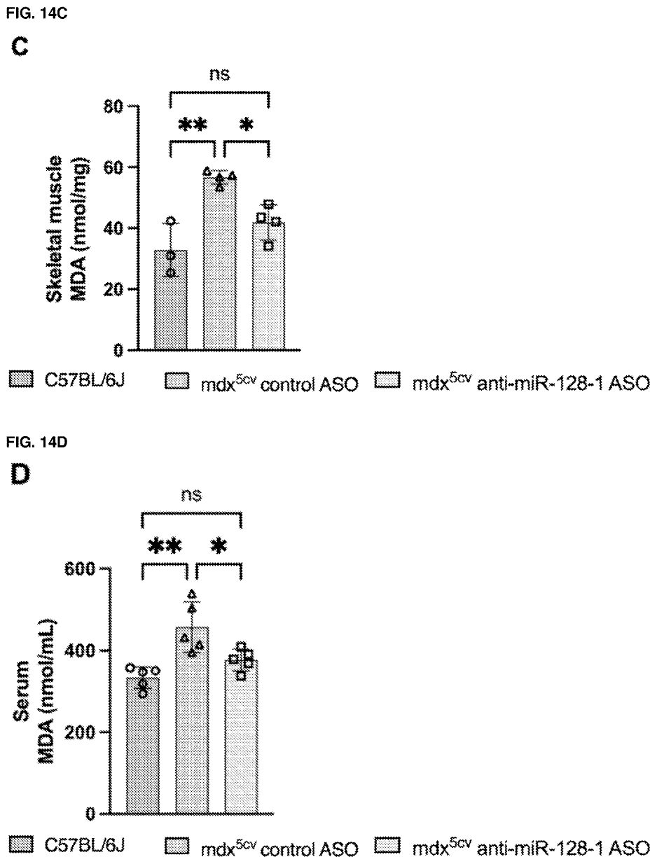

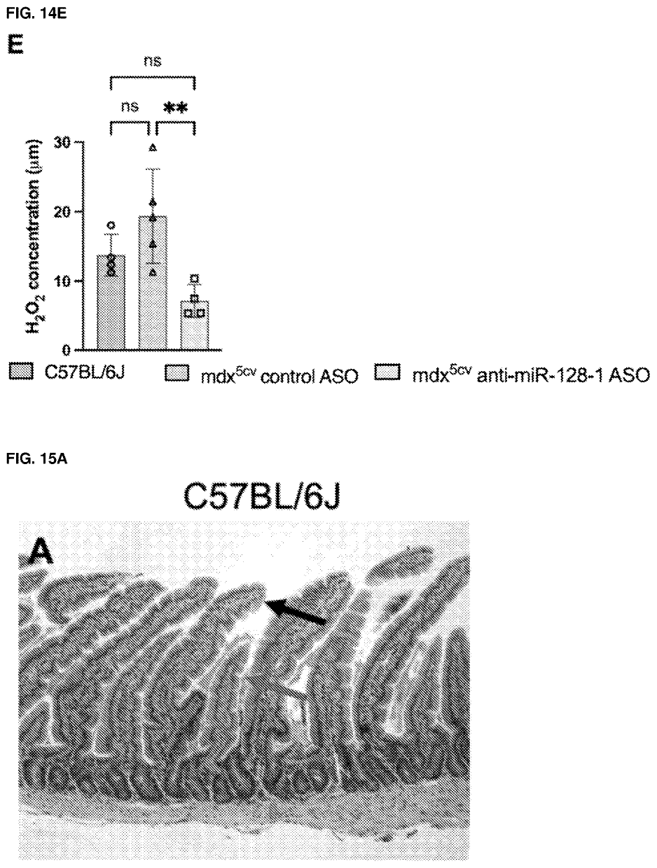

FIG. 14. Effect of anti-miR-128-1 ASO on inflammation, ferroptosis, and oxidative stress were examined. (A) TNFa, IL-1b and IL6 expression level were measured by RT-qPCR. (B) Genes involved in ferroptosis were examined. (C and D) Lipid oxidation byproduct MDA were measured in the TA skeletal muscle and serum of all three experimental groups. (E) Levels of H₂O₂ were measured in the TA muscle.

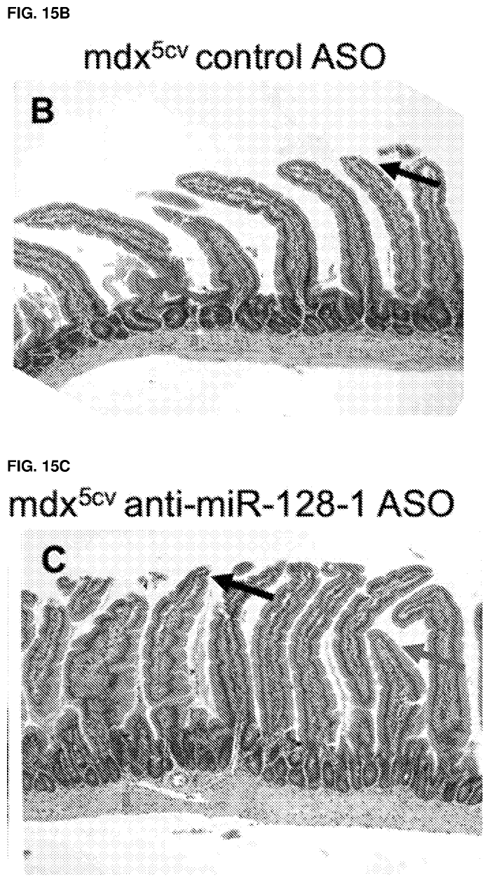

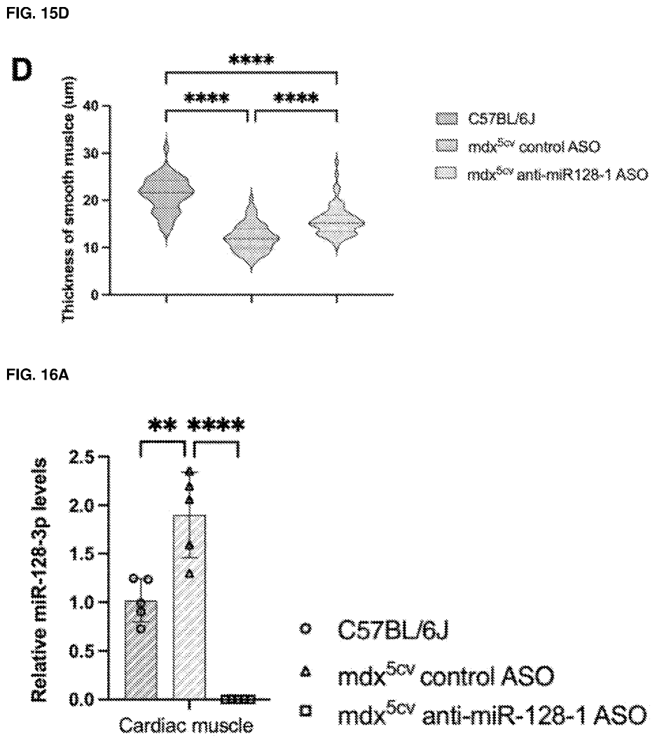

FIG. 15. Histology of the small intestine sections of WT and mdx^{5cv} mice treated with control ASO or anti-miR-128-3p ASO. (A-C) Representative H&E images of WT, mdx50Y control and treatment ASO groups. Red lines indicate the thickness of the intestinal wall, black arrows indicate the height of the small intestine villi and blue arrows indicate the depth of the small intestine crypt. (D) Quantification of the thickness of smooth muscle layer is plotted.

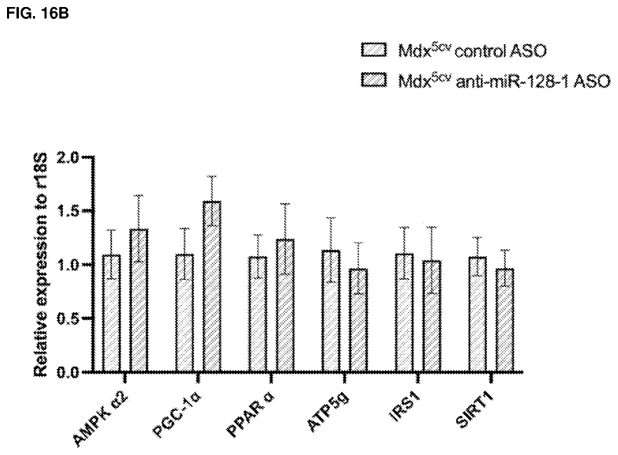

FIG. 16. Evaluation of miR-128-3p expression and its downstream targets in the cardiac muscle of mdx^{5cv} mice at 6 weeks of age. (A) miR-128-3p expression level in the heart of WT, mdx^{5cv} control ASO and treatment ASO group. (B) Gene expression profile of miR-128-3p downstream targets.

### Example 4: Myocardial Infarction (MI) and Heart Failure

miR-128-3p is a critical regulator of endogenous cardiac fibrosis and regeneration after myocardial infarction (MI). It was hypothesized that miR-128-3p may represent a therapeutic target to counter myocardial fibrosis and promote heart regeneration. To inhibit miR-128-3p, a state-of-the-art LNA ASO was used, which exhibits greatly improved stability and safety and which has shown high efficacy in several tissues, including skeletal muscle and heart.

### Anti-miR-128-3p LNA ASO Treatment Rescues Murine and Porcine Models of Cardiac Dysfunction

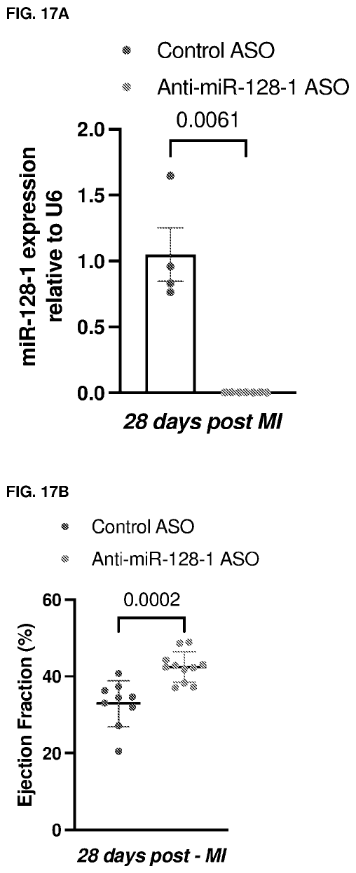

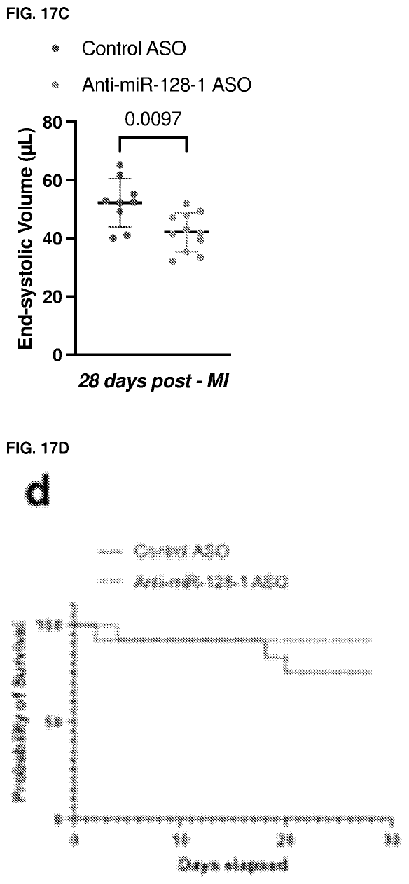

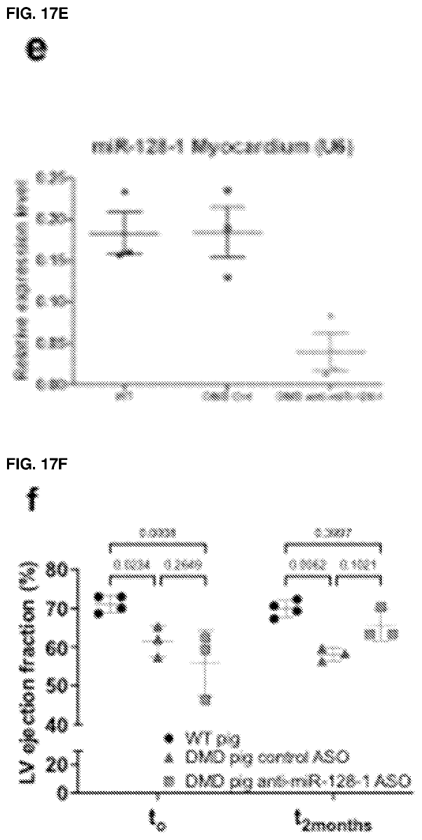

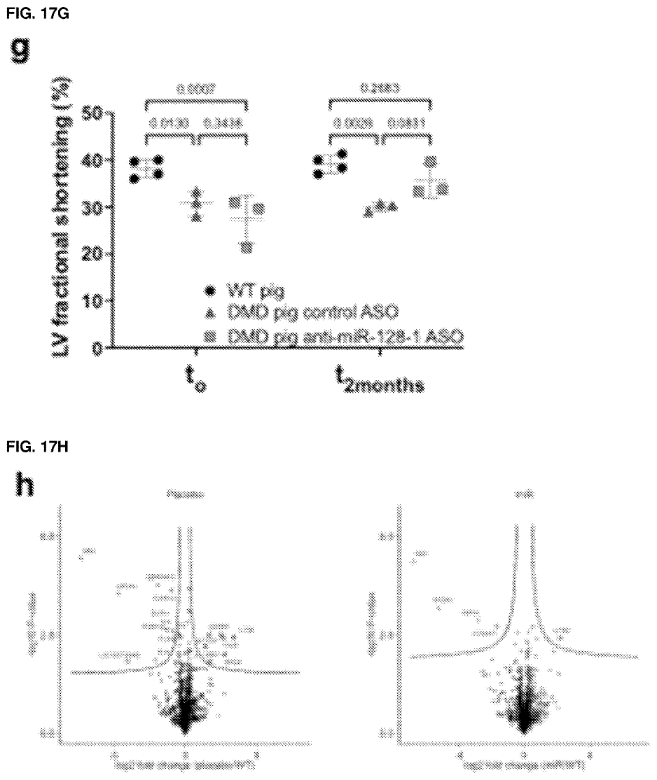

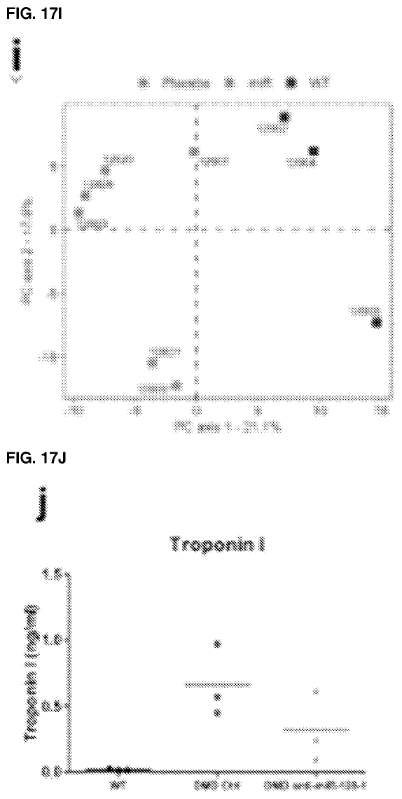

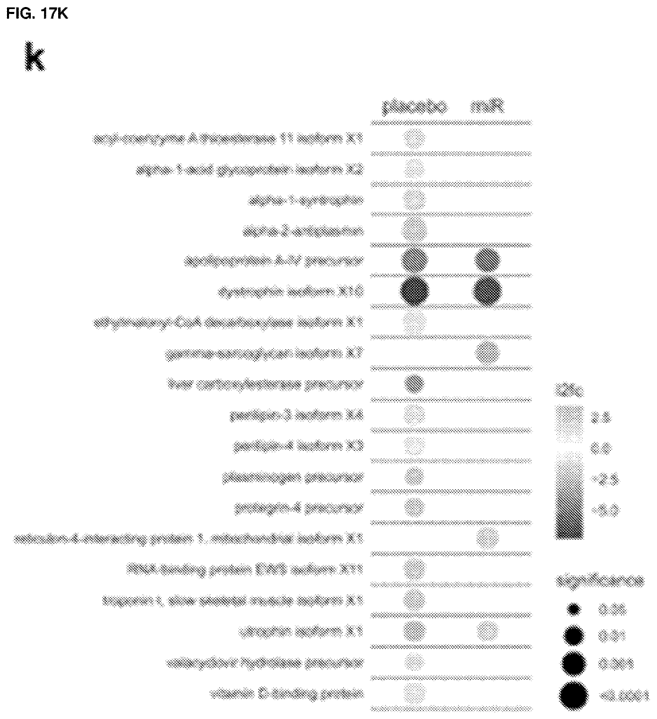

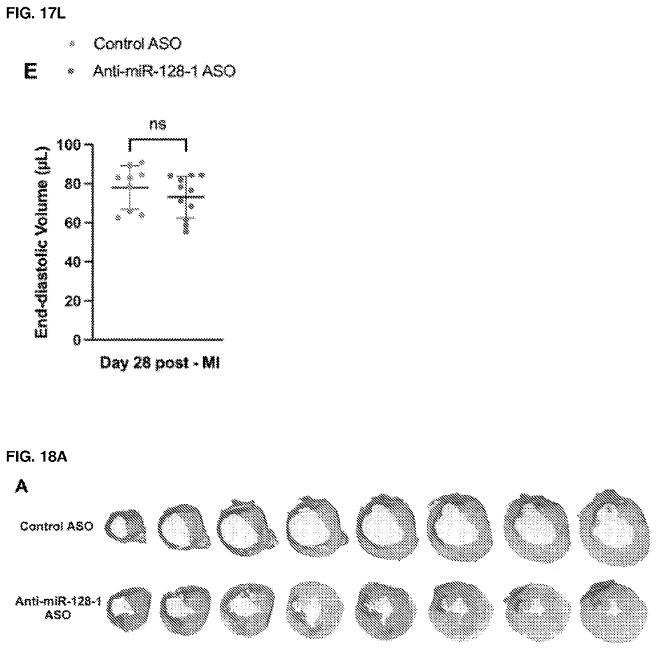

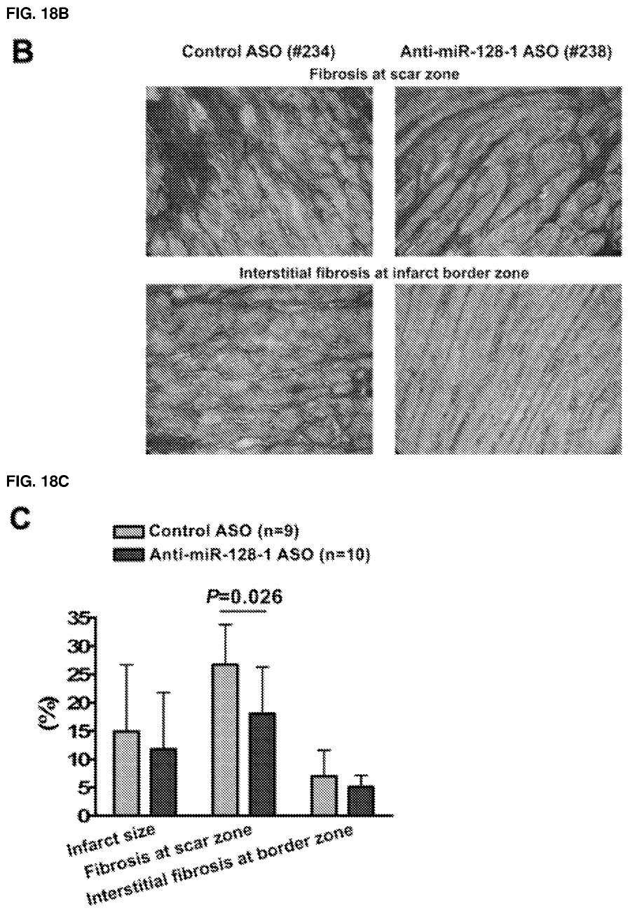

DMD patient mortality is ultimately the result of cardiac and respiratory failure. Because of the robust improvement in skeletal muscle function with anti-miR-128-3p LNA ASO treatment, it was sought to further explore whether treatment could also rescue cardiac insufficiency. The mdx^{5cv} model of DMD does not recapitulate the cardiac dysfunction observed in human patients. Therefore other models of heart failure were investigated. miR-128-3p cardiac-selective knockout and targeting via an AAV-decoy has been shown to improve cardiac function after myocardial infarction (MI) in mice. It was first examined whether the anti-miR-128-3p LNA ASO could improve cardiac function in a permanent ligation-induced heart failure model. Permanent ligation of the anterior descending coronary artery was performed, then mice were immediately injected subcutaneously with 10 mg/kg of LNA ASO. Injections were repeated weekly and tissues were harvested 28 days post myocardial infarction. Anti-miR-128-3p LNA ASO treatment resulted in complete knockdown of miR-128-3p expression in the heart 28 days post-myocardial infarction (FIG. 17A). miR-128-3p levels were very significantly knocked down, indicating high efficacy at targeting the cardiac tissue with once-weekly subcutaneously injection. Additionally, there is reduced fibrosis in the scar zone of the treatment group (FIG. 18).

Echocardiography was used to measure cardiac function before MI as well as 28 days later. These results revealed significant improvement in ejection fraction as well as a significant decrease in end-systolic volume after anti-miR-128-3p LNA ASO treatment, indicating marked improvement of cardiac function (FIG. 17B, C). Furthermore, mice treated with anti-miR-128-3p LNA ASO exhibited an increased probability of survival compared to those injected with scramble control LNA ASO (FIG. 17D).

Due to the striking improvement of cardiac function in mouse MI models, the therapeutic potential of the anti-miR-128-3p LNA ASO was further investigated in a very severe porcine model of DMD. Cardiac physiology of pigs better resembles that of humans, and the DMD^{Y/-} pig model exhibits marked cardiac dysfunction. To examine the effect of anti-miR-128-3p LNA ASO treatment, six-week-old pigs were treated with LNA ASO subcutaneously once-weekly at 5 mg/kg body weight for 2 months (FIG. 17E). After LNA ASO treatment, cardiac dysfunction in these DMD^{Y/-} pigs was markedly rescued as measured by LV ejection fraction and LV fractional shortening (FIG. 17F, G). Serum troponin I levels, a biomarker for myocardial infarction and cardiac cell death, were also rescued with LNA ASO treatment (FIG. 17J). To further elucidate the mechanism for rescuing cardiac dysfunction, mass-spectrometry based quantitative proteomic analysis of the myocardium was performed. Principal component analysis revealed a proteomic profile shift toward wild-type when DMD^{Y/-} pigs were treated with anti-miR-128-3p LNA ASO (FIG. 17I). Seventeen differentially regulated proteins are observed in the DMD disease state compared to wild-type pigs (FIG. 17H, K). However, DMD^{Y/-} pigs treated with anti-miR-128-3p LNA ASO show no expression pattern alteration in twelve of these seventeen proteins (FIG. 4h, k). Interestingly, troponin I, is elevated in DMD^{Y/-} pigs compared with wild-type, but not in the anti-miR-128-3p LNA ASO-treated DMD^{Y/-} pigs (FIG. 17K). Proteins with decreased abundance in DMD^{Y/-} versus wild-type samples belong to the functional clusters cell adhesion, regulation of response to wounding, protein maturation, protein activation and extracellular structure organization. Taken together, these results indicate that anti-miR-128-3p LNA ASO is an effective therapeutic approach for murine and porcine models of cardiac dysfunction.

FIG. 17. Anti-miR128-3p LNA ASO rescued cardiac dysfunction in mouse MI and preclinical DMD^{Y/-} pig models. (A), miR-128-3p levels were measured in the mouse MI model with LNA ASO treatment. (B, C) Ejection fraction and end-systolic volume were measured in the MI model. (D) Survival Kaplan-Meier estimator. (E) Endpoint miR-128-3p expression was measured in the myocardium of DMD^{Y/-} pigs treated with anti-miR-128-3p or control ASO. (F, G) Left ventricular fraction shortening and ejection fraction measured by echocardiography. (H) Volcano plot visualization of proteome alterations: proteins significantly altered in abundance in the DMD group (FDR<0.05) are colored in blue and red for downregulation and upregulation, respectively. (I) Principal component analysis of WT pig, DMD^{Y/-} pig placebo and anti-miR-128-3p LNA ASO experimental groups. (J) Serum level of Troponin I was measured. (h) WT n=4; DMD^{Y/-} n=3 for control and treated group. (K) Pathway analysis of differentially abundant proteins (FDR<0.05) in at least one condition are shown. (L) Echocardiographic evaluation of end-diastolic volume in the MI mouse model. n=9 for control ASO; n=11 Anti-miR-128-1 ASO.

FIG. 18. Evaluation of the fibrosis in the MI mouse model. (A) Picrosirius red staining is used to quantify the fibrotic tissue in the heart. Image J is used to quantify the area of fibrosis. (B) Fibrosis at the scar zone and interstitial fibrosis at the infarct border zone were measured. (C) Quantification of infarct size, fibrosis at scar zone and interstitial fibrosis at the border zone were measured and quantified. n=9 for control ASO; n=11 Anti-miR-128-1 ASO.

### Methods

Mouse Husbandry and Experiments. Dmd^{mdx-5Cv}/J mice (referred to herein as mdx^{5cv}) were purchased from the Jackson laboratory (Strain #002379) and were bred with C57BL/6J mice for at least 6 generations. Wild-type C57BL/6J mice were purchased from Jackson laboratory as controls. Muscle specific miR-128 knockout mice (MCK cre/miR-128^{f/f}/mdx^{5cv}) were generated from MCK-Cre mice (Jackson Labs Strain #006405) crossed with miR-128-1^{f/f} mice and bred to mdx^{5cv} (MCK cre/miR-128^{f/f}/mdx^{5cv}). mdx^{5cv} mice with whole body miR-128 knockout (CMV cre/miR-128^{f/f}/mdx^{5cv}) were generated using CMV-Cre mice (Jackson Labs Strain #006054). Wild type and mdx^{5cv} mice were injected with once weekly scramble control or anti-miR-128-3p ASO starting at 3 weeks of age. Behavioral tests were performed at 6 weeks and 9 weeks. Tissues and blood were harvested at the end of 6 weeks and 9 weeks. The collected blood samples were centrifuged at 2,000 rpm for 10 mins to obtain serum. Skeletal muscle tissues are used for morphology and molecular analysis. All experimental procedures were conducted in accordance with IACUC regulations and were approved by University of California Berkeley's IACUC (AUP #2018-10-11513-1). All protocols conform to federal regulations, the National Research Council Guide for the Care and Use of Laboratory Animals, and the Public Health Service Policy on Humane Care and Use of Laboratory Animals.

Pig Husbandry and Analysis All pig experiments were performed according to the German Animal Welfare Act and Directive 2010/63/EU on the protection of animals used for scientific purposes and were approved by the responsible animal welfare authority (Government of Upper Bavaria; permission 55.2-1-54-2532-163-2014). All pigs were kept in a specified pathogen-free environment (Center for Innovative Medical Models) at the Ludwig-Maximilian University Munich. Food and water were provided ad libitum. DMD and WT pigs were from the same litter. To standardize the experimental groups, all piglets in the placebo and treatment groups were produced by breeding the same boar with heterozygous carrier sows (DMD^{+/-}).

The generation of the porcine model with a deletion of DMD exon 52 has been described in detail elsewhere (Stirm, Fonteyne et al. 2021).

The first cardiac ultrasound examination and blood sampling was performed at age 5 weeks (one week after weaning). The first injection took place one week later. Three DMD pigs each were randomly assigned to the treatment and placebo groups and received 5 mg per kg body weight of an LNA ASO against miR-128-3p or a random LNA sequence, respectively, weekly for 2 months. The injections were performed subcutaneously into the skin fold just behind the auricle. Further blood samples were taken two weeks after the first injection and after two months, coinciding with the final cardiac ultrasound examination, necropsy and tissue sampling.

Locked nucleic acid antisense oligonucleotides (LNA ASOs). ASO targeting microRNA miR-128 were designed and modified for stability and efficacy. The sequences are 14-mer, ACcggTTCacTgTG (SEQ ID NO:1). The LNA ASO is 100% chemically synthesized by IDT DNA, and is non-hazardous, non-toxic and non-infectious. It is also at pharmaceutical grade, and endotoxin free.

Muscle Function Behavioral Tests. All mice were exercised twice per week for three weeks using treadmill running at 9 m/min for 30 minutes (Kaczor et al., 2007; Hudecki et al., 1993). Behavioral tests of muscle function including the passive wirehanging, four-limb wire hanging, and treadmill exhaustion test were performed according to the Treat NMD Neuromuscular Network standard operating procedures. For the treadmill exhaustion test, mice were placed on the belt of a six-lane motorized treadmill (Exer 3/6 Treadmill; Columbus Instruments, Columbus, OH) supplied with shocker plates. The treadmill was run at an inclination of 0° at 5 m/min for 5 min, after which the speed was increased 1 m/min every minute. The test was stopped when the mouse remained on the shocker plate for 20 s without attempting to re-engage the treadmill, and the time to exhaustion was determined. The distance is then calculated based on the speed and running time.

Creatine kinase, Alanine transaminase (ALT), Aspartate aminotransferase (AST), Blood Urea Nitrogen (BUN), Creatinine Measurements. Serum levels of creatine kinase, ALT, AST, BUN, and creatinine were measured using commercially available kits.

RT-qPCR GA skeletal muscles were used for total RNA extraction. The levels of miR-128 were measured using probes from Thermo Scientific ( ). U6 was used as housekeeping control.

Reverse transcriptase kits from BioRad were used to generate cDNA, and then gene expression was measured by qPCR.

RNAseq analysis. Total RNA was extracted using Qiagen Kit (catalog). cDNA libraries were constructed using the Stranded mRNA-seq kit (KAPA) from 1 μg of total RNA from GA muscle tissue of mice according to the manufacturer's protocol. Libraries were sequenced on the NovaSeq 6000 (Novogene) targeting 40 million read pairs and extending 150 cycles with paired end reads. STAR aligner was used to map sequencing reads to transcripts in the mouse mm10 reference genome. Read counts for individual transcripts were produced with HTSeq-count followed by the estimation of expression values and detection of differentially expressed transcripts using EdgeR. Differentially expressed genes were defined by at least 2-fold change with FDR less than 0.01.

Immunohistochemistry and Histology. TA muscle tissue was embedded in OCT and flash frozen in liquid nitrogen. Muscle was sectioned to 10 μM sections. Hematoxylin and eosin staining was performed. Muscle fiber size was measured from the H&E sections using Image J. Picosirius red staining was performed to stain for fibrotic tissue. IgG, IgM, and IgA were used to stain necrotic cells. For quantification, three images were analyzed for each sample to generate a combined average.

Blood collection and clinical chemistry. Blood collection took place from the right jugular vein, using Serum Monovettes® (Sarstedt, Nümbrecht, Germany). Serum Monovettes® were kept at room temperature for 30 min for clotting, followed by centrifugation at 1800 g for 20 min at 4° C. Serum was aliquoted and stored at −80° C. until further processing (storage period did not exceed 6 months). Creatine kinase values were determined using the CKL ACN 057 kit on a Cobas 311 Analyser System (Sridhar, Roche et al.). Troponin I levels were measured by CMIA (Alinity, Abbott, Illinois, U.S.A) in the laboratory of SYNLAB.vet GmbH Augsburg.

Echocardiography. For the echocardiography, pigs were sedated with 20 mg/kg ketamine (Ursotamin®, Serumwerke Bernburg, Bernburg, Germany) and 2 mg/kg azaperone (Azaporc®, Serumwerke Bernburg), followed by an anesthesia with 4 mg/kg/h of propofol (Propofol 2%, Fresenius Kabi, Bad Homburg, Germany). After sufficient depth of anesthesia has been achieved, pigs were placed in right lateral recumbency and heart function was measured by standard 2D transthoracic echocardiography (Esaote MyLab X8). Left ventricular ejection fraction and fractional shortening was determined by M-mode method. All measurements were performed by the same investigator. The genotypes could not be blinded because of the significant differences, but measurements of the placebo and treatment groups were blinded to the investigator.

Sample preparation for proteome analysis. Heart and skeletal muscle tissue samples were snap-frozen in liquid nitrogen and were cryo-pulverized using a CP02 Automated Dry Pulverizer (Covaris, Woburn, MA, USA) according to the manufacturer's instructions. Tissue powder was lysed in 8 M urea/0.5 M NH4HCO3 by ultrasonication (18 cycles of 10 s) using a Sonopuls HD3200 (Bandelin, Berlin, Germany). Proteins were quantified with Pierce 660 nm Protein Assay (Thermo Fisher Scientific, Rockford, IL, USA). Twenty micrograms of protein were digested with Lys-C (FUJIFILM Wako Chemicals Europe GmbH, Neuss, Germany) for 4 h and subsequently with modified porcine trypsin (Promega, Madison, WI, USA) for 16 h at 37° C.

Nano-liquid chromatography (LC)-tandem mass spectrometry (MS) analysis and bioinformatics. 1 μg of the digest was injected on an UltiMate 3000 nano-LC system coupled online to a Q-Exactive HF-X instrument (Thermo Fisher Scientific). Samples were transferred to a PepMap 100 C18 trap column (100 μm×2 cm, 5 μM particles, Thermo Fisher Scientific) and separated on an analytical column (PepMap RSLC C18, 75 μm×50 cm, 2 μm particles, Thermo Fisher Scientific) at 250 nl/min with an 80-min gradient of 5-20% of solvent B followed by a 9-min raise to 40%. Solvent A consisted of 0.1% formic acid in water and solvent B of 0.1% formic acid in acetonitrile. MS spectra were acquired using a top 15 data-dependent acquisition method on a Q Exactive HF-X mass spectrometer. Raw files were processed with Maxquant (v.1.6.7.0) [1], using its built-in search engine Andromeda [2] and the NCBI RefSeq Sus scrofa database (v.7-5-2020). Protein intensities were normalized using the MaxLFQ approach [3]. Statistical analysis and visualization were done using Perseus [4] and R framework [5]. Proteins detected in all replicates of at least one condition were kept for quantitative analysis. Missing values were imputed by Perseus default parameters. The volcano plots were generated using two-tailed Student's t-test and permutation-based FDR cut-off of 0.05, together with an s0-parameter of 0.1 to additionally consider fold changes [6].

### REFERENCES

1 Wagschal, A. et al. Genome-wide identification of microRNAs regulating cholesterol and triglyceride homeostasis. Nat Med 21, 1290-1297 (2015).
2 Wang, L. et al. A MicroRNA Linking Human Positive Selection and Metabolic Disorders. Cell 183, 684-701 e614 (2020).
3 Kuno, A. & Horio, Y. SIRT1: A Novel Target for the Treatment of Muscular Dystrophies. Oxid Med Cell Longev 2016, U.S. Pat. No. 6,714,686 (2016).
4 Miura, P. et al. Pharmacological activation of PPARbeta/delta stimulates utrophin A expression in skeletal muscle fibers and restores sarcolemmal integrity in mature mdx mice. Hum Mol Genet 18, 4640-4649 (2009).
5 Ljubicic, V. & Jasmin, B. J. AMP-activated protein kinase at the nexus of therapeutic skeletal muscle plasticity in Duchenne muscular dystrophy. Trends Mol Med 19, 614-624 (2013).
6 Pauly, M. et al. AMPK activation stimulates autophagy and ameliorates muscular dystrophy in the mdx mouse diaphragm. Am J Pathol 181, 583-592 (2012).
7 Vieira, N. M. et al. Jagged 1 Rescues the Duchenne Muscular Dystrophy Phenotype. Cell 163, 1204-1213 (2015).
8 von Maltzahn, J., Renaud, J. M., Parise, G. & Rudnicki, M. A. Wnt7a treatment ameliorates muscular dystrophy. Proc Natl Acad Sci USA 109, 20614-20619 (2012).
9 Ambros, V. The functions of animal microRNAs. Nature 431, 350-355 (2004).
10 Bartel, D. P. MicroRNAs: target recognition and regulatory functions. Cell 136, 215-233 (2009).
11 Zaharieva, I. T. et al. Dystromirs as serum biomarkers for monitoring the disease severity in Duchenne muscular Dystrophy. PLOS One 8, e80263 (2013).
12 Najafi-Shoushtari, S. H. et al. MicroRNA-33 and the SREBP host genes cooperate to control cholesterol homeostasis. Science 328, 1566-1569 (2010).
13 Motohashi, N., Alexander, M. S., Casar, J. C. & Kunkel, L. M. Identification of a novel microRNA that regulates the proliferation and differentiation in muscle side population cells. Stem Cells Dev 21, 3031-3043 (2012).
14 Motohashi, N. et al. Regulation of IRS1/Akt insulin signaling by microRNA-128a during myogenesis. J Cell Sci 126, 2678-2691 (2013).
15 Amor, F. et al. Cholesterol metabolism is a potential therapeutic target in Duchenne muscular dystrophy. J Cachexia Sarcopenia Muscle 12, 677-693 (2021).
16 Pambianco, S. et al. Reversal of Defective Mitochondrial Biogenesis in Limb-Girdle Muscular Dystrophy 2D by Independent Modulation of Histone and PGC-1alpha Acetylation. Cell Rep 17, 3010-3023 (2016).
17 Maciotta, S. et al. Hmgb3 Is Regulated by MicroRNA-206 during Muscle Regeneration. Plos One 7 (2012). e43464
10.1371/journal.pone.0043464
18 Rottiers, V. et al. Pharmacological inhibition of a microRNA family in nonhuman primates by a seed-targeting 8-mer antimiR. Sci Transl Med 5, 212ra162 (2013).
19 Huang, W. et al. Loss of microRNA-128 promotes cardiomyocyte proliferation and heart regeneration. Nat Commun 9, 700 (2018).
20 Ruiz-Velasco, A. et al. Targeting mir128-3p alleviates myocardial insulin resistance and prevents ischemia-induced heart failure. Elife 9 (2020).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An inhibitory nucleic acid comprising the nucleotide sequence:
a)
(SEQ ID NO: 1)
5′-ACCGGTTCACTGTG-3′;
or
b)
(SEQ ID NO: 2)
5′-GACCGGTTCACTGT-3′;
or
c)
(SEQ ID NO: 3)
5′-AGACCGGTTCACTGTG-3′,
wherein the inhibitory nucleic acid comprises one or more locked nucleic acids (LNA).

2. The inhibitory nucleic acid of claim 1, wherein the inhibitory nucleic acid comprises:
i) a modified backbone; and/or
ii) one or more 5-methyl deoxycytosine residues.

3. The inhibitory nucleic acid of claim 1 or claim 2, wherein the inhibitory nucleic acid has a length of from 14 nucleotides to 20 nucleotides.

4. The inhibitory nucleic acid of any one of claims 1-3, wherein the inhibitory nucleic acid comprises the nucleotide sequence depicted in FIG. 1A and designated “NRC0090.”

5. The inhibitory nucleic acid of any one of claims 1-3, wherein the inhibitory nucleic acid comprises the nucleotide sequence depicted in FIG. 1A and designated “NRC-0091.”

6. The inhibitory nucleic acid of any one of claims 1-3, wherein the inhibitory nucleic acid comprises the nucleotide sequence depicted in FIG. 1A and designated “NRC-0119.”

7. A pharmaceutical composition comprising:
a) an inhibitory nucleic acid of any one of claims 1-6; and
b) a pharmaceutically acceptable excipient.

8. The pharmaceutical composition of claim 7, wherein the pharmaceutically acceptable excipient comprises one or more lipids.

9. The pharmaceutical composition of claim 7, wherein the pharmaceutically acceptable excipient comprises poly(amidoamine), poly(propyleneimine), or poly(L-lysine).

10. A lipid nanoparticle comprising:
a) an inhibitory nucleic acid of any one of claims 1-6; and
b) a pharmaceutically acceptable excipient.

11. A method of treatment, the method comprising administering to an individual in need thereof an effective amount of an inhibitory nucleic acid of any one of claims 1-6, a pharmaceutical composition of any one of claims 7-9, or a lipid nanoparticle of claim 10.

12. A method of treating a metabolic disorder in an individual, the method comprising administering to the individual an effective amount of an inhibitory nucleic acid of any one of claims 1-6, a pharmaceutical composition of any one of claims 7-9, or a lipid nanoparticle of claim 10.

13. The method of claim 12, wherein the metabolic disorder is insulin resistance, hyperglycemia, type 2 diabetes mellitus, obesity, fatty liver disease, glucose intolerance, hyperinsulinemia, metabolic syndrome, or hypertension.

14. The method of claim 12, wherein the metabolic disorder comprises insulin resistance.

15. The method of claim 12, wherein the metabolic disorder comprises metabolic syndrome.

16. The method of claim 12, wherein the metabolic disorder comprises type 2 diabetes mellitus.

17. The method of any one of claims 12-16, wherein the individual has a body mass index>30.0.

18. The method of any one of claims 12-17, wherein said administering results in a serum insulin level in a normal range.

19. The method of any one of claims 12-17, wherein said administering results in a blood glucose level in a normal range.

20. The method of any one of claims 12-19, further comprising administering at least one additional therapeutic agent.

21. The method of claim 20, wherein the at least one additional therapeutic agent is insulin, an insulin analog, a biguanidine, or a thiazolidinedione.

22. The method of any one of claims 11-20, wherein said administering is via oral administration.

23. A method of treating muscular dystrophy in an individual, the method comprising administering to the individual an effective amount of an inhibitory nucleic acid of any one of claims 1-6, a pharmaceutical composition of any one of claims 7-9, or a lipid nanoparticle of claim 10.

24. The method of claim 23, wherein the muscular dystrophy is Duchenne muscular dystrophy or Becker muscular dystrophy.
